(19) United States
(12) Patent Application Publication
(10) Pub. No.: US 2009/0076584 A1
(43) Pub. Date: Mar. 19, 2009

(54) APPARATUS AND METHODS FOR DEPLOYMENT OF MULTIPLE CUSTOM-LENGTH PROSTHESES

(75) Inventors: Bryan Mao, San Francisco, CA (US); Pablo Acosta, Newark, CA (US)

Correspondence Address: TOWNSEND AND TOWNSEND AND CREW, LLP;(CLIENT NO 021629-000000), TWO EMBARCADERO CENTER, 8TH FLOOR, SAN FRANCISCO, CA, 94111-3834, US

(73) Assignee: Xtent, Inc., Menlo Park, CA (US)

(21) Appl. No.: 11/857,562

(22) Filed: Sep. 19, 2007

(51) Int. Cl. A61F 2/06

(52) U.S. Cl. 623/1.11

(57) ABSTRACT

A catheter for delivering a prosthesis to a target treatment site comprises an inner shaft and an expansion member coupled to the inner shaft. A plurality of radially expandable prosthetic segments are positionable over the expansion member and they are releasably interlocked with one another while unexpanded. Adjacent pairs of prosthetic segments may decouple from one another upon radial expansion of the distal prosthetic segment in the adjacent pair while the proximal segment in the pair remains at least partially unexpanded. The catheter also includes an outer sheath that is axially movable and positionable at least partially over the prosthetic segments to constrain expansion of a selectable number of segments. A segment mover is coupled to at least one of the prosthetic segments and is axially movable so as to retract one or more of the segments when the one or more prosthetic segments are unexpanded.

### BACKGROUND OF THE INVENTION

1. Field of the Invention

This invention relates generally to medical apparatus and methods, and more specifically to vascular catheters, stents and stent delivery systems for use in the coronary arteries and other vessels.

Stenting is an important treatment option for patients with vascular occlusive disease. The stenting procedure involves placing a tubular prosthesis at the site of a lesion, typically within a diseased coronary artery. The procedure is performed in order to maintain the patency of the artery and is often performed after a primary treatment such as angioplasty. Early stent results suffered from high rates of restenosis, i.e. the tendency for the stented coronary artery to become re-occluded following implantation of the stent. Recently however, restenosis rates have decreased substantially, due in part to drug eluting stents as well as other improvements in stent delivery methods and stent technology. As a result, the number of stent related procedures being performed worldwide continues to dramatically increase.

Stents are typically either self-expanding or balloon expandable and they are delivered to the coronary arteries using long, flexible vascular catheters typically inserted percutaneously through the patient's femoral artery. For self-expanding stents, the stent is simply released from the delivery catheter and then it resiliently expands into engagement with the vessel wall. For balloon expandable stents, the stents are typically mounted over a balloon on the delivery catheter. As the balloon expands, the stents also expand and deform to a desired diameter, whereupon the balloon is deflated and removed, leaving the stent or stents in place.

Current stent delivery technology suffers from a number of drawbacks which can make delivery of stents challenging. In particular, current stent delivery catheters often employ stents having fixed lengths. The proper selection of a fixed length stent requires accurate knowledge of the lesion length being treated. While lesion length may be measured prior to stent deployment using angiography and fluoroscopy, these measurements are often inaccurate. Thus, if an incorrectly sized stent is introduced to a treatment site, it must be removed from the patient along with the delivery catheter and replaced with a different device having the correct stent size. This prolongs the procedure, increases waste and results in a more costly procedure.

The use of “custom length” stents as an alternative to fixed length stents has been proposed. One such approach for providing a custom length stent has been to use segmented stents for treatment in which only some of the stents are deployed for treatment. Several exemplary systems are described in several copending, commonly assigned applications which are listed below. In these systems, the stent segments are deployed by selective advancement over the delivery catheter. After delivering an initial group of segments, the catheter may be repositioned to a new treatment site and a further group of segments can then be deployed. These systems enable treatment of multiple lesions with a single device and may contain up to fifty segments. While this technology represents a significant improvement over earlier stent delivery systems, these delivery systems can be complex to operate and may require higher forces or torques to be exerted during operation due to friction from stent selection and deployment mechanisms (sometimes referred to as “stent valves” or “stent separators”) in these systems. Thus it would be desirable to provide a stent delivery system that allows deployment of multiple customized length prostheses that is easier to operate by requiring less force or torque to actuate during use. Another challenge with existing “custom length” stent delivery systems is that to deliver multiple stent segments to multiple lesion sites requires an intricate delivery system that can be somewhat complex to use and in some situations can occasionally damage some of the stents. Thus, a simpler, more reliable delivery system having fewer components while still permitting length customization is also desirable. Additionally, therapeutic agents are often coupled to stents to provide localized drug delivery at the site of a lesion. In some instances, the custom length stent delivery systems can damage the therapeutic agent coating the stent during stent selection and deployed. Therefore, it is also desirable to provide a stent delivery system that is less likely to damage any therapeutic agents carried by the stents during deployment.

For the above reasons as well as others, it would be desirable to provide improved prosthetic stents and delivery catheters. It is also desirable to provide a delivery system that is flexible and can track torturous vessels and that has a simple construction and is less costly and easy to use in deploying a selectable number of stent segments to a single treatment site.

2. Description of the Background Art

Patents describing catheters for delivering multiple segmented stents include U.S. Pat. Nos. 7,182,779 and 7,137,993. Other publications describing catheters for delivering multiple segmented stents include: U.S. Patent Publication Nos. 2006/0282150, 2006/0282147, 2004/0098081, 2005/0149159, 2005/0038505, 2004/0186551 and 2003/013266. Prior related unpublished co-pending U.S. patent applications include U.S. patent Ser. Nos. 11/344,464, filed Jan. 30, 2006 (Attorney Docket No. 021629-003500US), entitled “Apparatus and Methods for Deployment of Custom-Length Prostheses”; Ser. No. 11/687,885, filed Mar. 19, 2007 (Attorney Docket No. 021629-003610US), entitled “Apparatus and Methods for Deployment of Linked Prosthetic Segments”; and Ser. No. 11/462,951, filed Aug. 7, 2006 (Attorney Docket No. 021629-004100US), entitled “Custom Length Stent Apparatus.” The full disclosures of each of these patents, publications and applications are incorporated herein by reference.

### BRIEF SUMMARY OF THE INVENTION

The invention provides apparatus and methods for delivering a prosthesis into body lumens such as an artery. The prosthesis often is composed of a plurality of prosthetic segments or stent segments.

In a first aspect of the present invention, a catheter for delivering a prosthesis to a target treatment site comprises an inner shaft having a proximal end and a distal end. An expansion member is coupled to the inner shaft near the distal end and a plurality of radially expandable prosthetic segments are positionable over the expansion member. The plurality of radially expandable prosthetic segments are releasably interlocked with one another while unexpanded and adjacent pairs of prosthetic segments are adapted to decouple from one another upon radial expansion of the distal prosthetic segment of the adjacent pair while a proximal prosthetic segment of the adjacent pair remains at least partially unexpanded. The catheter also includes an outer sheath that is axially movable relative to the expansion member and the sheath is positionable at least partially over the plurality of radially expandable prosthetic segments to constrain expansion of a selectable number thereof. A segment mover is axially movable relative to the expandable member and also is coupled or releasably interlocked with at least one of the plurality of radially expandable prosthetic segments. The segment mover is also adapted to retract one or more of the plurality of radially expandable prosthetic segments proximally relative to the expansion member when the one or more prosthetic segments is unexpanded.

In another aspect of the present invention, a catheter for delivering a prosthesis to a target treatment site comprises an inner shaft having a proximal end, a distal end, a proximal section with a first diameter and a distal section with a second diameter larger than the first. A ramp is between the proximal and distal sections and the ramp is adapted to provide a transition from the first diameter to the second diameter. An expandable member coupled to the inner shaft is adjacent to the distal end and a plurality of radially expandable prosthetic segments are positionable over the expandable member. The plurality of radialy expandable prosthetic segments are releasably interlocked with one another while unexpanded and adjacent pairs of the prosthetic segments are adapted to decouple from one another upon radial expansion of a distal prosthetic segment of the adjacent pair while a proximal prosthetic segment of the adjacent pair remains at least partially unexpanded. An outer sheath is axially movable relative to the expansion member and is positionable at least partially over the plurality of radially expandable prosthetic segments. A segment mover is axially movable relative to the expandable member and is coupled to at least one of the plurality of radially expandable prosthetic segments. The segment mover is adapted to retract one or more of the plurality of radially expandable prosthetic segments proximally relative to the expandable member when the one or more prosthetic segments are unexpanded. Axially moving a prosthetic segment over the ramp partially expands the prosthetic segment from a first unexpanded diameter to a second partially expanded diameter. The prosthetic segment is adapted to decouple from an adjacent prosthetic segment in the partially expanded diameter.

The ramp may have a proximal diameter that is substantially similar to the first diameter of the proximal section of the inner shaft. The ramp may have a distal diameter that is substantially similar to the second diameter of the distal section of the inner shaft. The outer sheath may comprise a resilient section near its distal end and the resilient section may be adapted to axially contract as the expandable member expands and the resilient section can expand substantially back to its uncontracted configuration as the expandable member is contracted or deflated. The resilient section may comprise a bellows or a spring. The resilient section is adapted to allow a balloon taper to form when the resilient section axially contracts. The expansion member may be expandable and can be a balloon.

Sometimes the catheter may also comprise a control mechanism that is coupled to the proximal end of the inner shaft. The control mechanism may have an actuator that is adapted to move the outer sheath or the segment mover.

Each of the prosthetic segments may have at least one locking element on a distal end thereof and at least one receptacle region on a proximal end. The receptacle region is configured to capture the locking element of an adjacent prosthetic segments when the prosthetic segments are unexpanded so as to constrain axial movement of one prosthetic segment away from the other prosthetic segment. Radial expansion of the prosthetic segment may cause a change in shape of the receptacle region or a change in position of the receptacle relative to the adjacent prosthetic segment, therefore, upon radial expansion of a prosthetic segment the receptacle region releases the locking element of an adjacent unexpanded prosthetic segment. The receptacle region often may be disposed between two locking elements on the same prosthetic segment. Each locking element may define a receptacle region that captures a locking element on an adjacent prosthetic segment.

At least one of the plurality of prosthetic segments may comprise an axially extending member that has an enlarged head region that is adapted to releasably interlock with a receptacle on an adjacent prosthetic segment. The enlarged head region may be triangular shaped. The receptacle region may be formed by a space between two axially extending members that each have an enlarged head region on an adjacent prosthetic segment. The receptacle often may widen upon expansion of the adjacent prosthetic segment. At least one of the plurality of prosthetic segments may comprise one or more arms axially extending therefrom and the arm may be adapted to releasably interlock with one or more arms that axially extend from an adjacent prosthetic segment. One or more of the axially extending arms may release from one or more arms axially extending from an adjacent prosthetic segment upon expansion of the adjacent prosthetic segment. The arms may be T-shaped or L-shaped.

The plurality of prosthetic segments may also carry a therapeutic agent such as an anti-restenosis agent, that is adapted to be released therefrom.

The inner shaft of the catheter may have a lumen that is disposed between the proximal and distal ends and the lumen may be able to accommodate a guidewire. The outer sheath may comprise a resilient section near a distal end thereof and the resilient section may be able to expand as a prosthetic segment positioned therein is expanded by the expansion member. The resilient section may have a portion that is radiopaque and the resilient section may be able to collapse substantially back to its unexpanded configuration as the expansion member collapses. The resilient section may crimp any portion of a prosthetic segment that is disposed thereunder back to a substantially unexpanded configuration when the resilient section collapses. The resilient section may comprise a plurality of fingers that axially extend away from the outer sheath. Sometimes the fingers may be hinged or they may have a plurality of apertures extending therethrough.

The sheath may be adapted to collapse a partially expanded prosthetic segment back to a substantially unexpanded configuration by retraction of the partially expanded prosthetic segment into the sheath. The outer sheath may comprise a flange near a distal end thereof and the flange is adapted to engage a prosthetic segment as the outer sheath is retraced proximally thereby also retracting the prosthetic segment therewith. Sometimes the outer sheath may also be reinforced so as to help restrain expansion of at least a portion of the expansion member. The catheter may also include a crimping member that is positionable over a prosthetic segment and that is adapted to crimp the prosthetic segment to a reduced diameter when the crimping member is disposed thereover. The crimping member may be disposed over the outer sheath and may comprise an o-ring or a tube slidably movable over the outer sheath.

Sometimes the catheter may also comprise an automatic separation mechanism that is coupled to the prosthetic segment moving tube such that the moving tube is adapted to retract as the expansion member expands or contracts, thereby separating the unselected prosthetic segments from the selected number of prosthetic segments. The automatic separation mechanism may comprise a piston mechanism or an actuator that is coupled to the stent moving tube. The expansion member and the automatic separation mechanism may be fluidly coupled together.

The prosthetic segment mover may be adapted to advance the plurality of prosthetic segments distally as the segment mover is advanced distally. The segment mover may also be adapted to retract the interlocked plurality of prosthetic segments proximally as the mover is retraced proximally. The segment mover may comprise a plurality of fingers that axially extend therefrom and they are adapted to releasably interlock with fingers that axially extend from an adjacent prosthetic segment. The segment mover may also comprise an axially extending member that has an enlarged head region that is adapted to releasably interlock with a receptacle on an adjacent prosthetic segment.

Sometimes the catheter may also comprise a proximal section of the inner shaft having a first diameter and a distal section of the inner shaft having a second diameter that is larger than the first diameter. The catheter may include a ramp that is between the proximal and distal sections and the ramp is adapted to provide a transition from the first diameter to the second diameter. Axially moving a prosthetic segment over the ramp partially expands the prosthetic segment from a first unexpanded diameter to a second partially expanded diameter and the prosthetic segment is adapted to decouple from adjacent prosthetic segments in the partially expanded diameter.

In another aspect of the present invention, a method for deploying a prosthesis into a treatment site in a body lumen comprises advancing a delivery catheter to the treatment site. The delivery catheter has a plurality of radially expandable prosthetic segments that are disposed thereon and that are also at least partially covered by a sheath. The plurality of radially expandable prosthetic segments are releasably interlocked with one another while unexpanded. Selecting a number of prosthetic segments to deploy into the body lumen allows the selected number of prosthetic segments to have a length that substantially traverses a length of a lesion at the treatment site. The selected number is less than the total number of prosthetic segments on the catheter. Radially expanding the selected number of prosthetic segments allows the selected number of prosthetic segments to decouple from at least one other of the prosthetic segments which remains at least partially unexpanded on the delivery catheter during expansion of the selected number of prosthetic segments. Retracting within the catheter moves the at least one other unexpanded prosthetic segment away from the selected number of expanded prosthetic segments. The delivery catheter is then removed from the treatment site with the at least one other unexpanded prosthetic segment disposed thereon and the selected number of expanded prosthetic segments are implanted at the treatment site.

Sometimes the prosthetic segments may be expanded by expanding an expansion member on the delivery catheter and the at least one other unexpanded prosthetic segment is retracted after the expansion member is expanded. A middle prosthetic segment may be disposed between the selected number of prosthetic segments to be expanded and the at least one other prosthetic segment constrained from expansion. The middle prosthetic segment may have a proximal portion which is unexpanded and a distal portion which is partially expanded when the selected number of prosthetic segments are expanded. A distal end of the middle prosthetic segment may be released from the selected number of prosthetic segments upon the expansion thereof and a proximal end of the middle prosthetic segment may remain connected to the at least one other prosthetic segments which remain unexpanded.

The step of retracting may comprise retracting the middle prosthetic segment into the sheath which crimps the distal portion of the middle prosthetic segment into an unexpanded shape. Selecting may comprise actuating a control mechanism adjacent to a proximal end of the delivery catheter. Selecting may also comprise moving the outer sheath so as to expose the selected number of prosthetic segments from the outer sheath.

Moving the outer sheath may comprise proximally retracting the outer sheath. Radially expanding the selected number of prosthetic segments may comprise inflating a balloon. The step of radially expanding may also comprise moving an expansion member under the selected number of prosthetic segments causing radial expansion thereof. The expansion member may comprise a substantially spherical head. The step of radially expanding may also comprise flaring a distal end of the outer sheath as the prosthetic segments expand.

The method may also include the step of radially contracting an expandable member such as by deflating a balloon. Radially contracting the expandable member may comprise collapsing a flared distal end of the outer sheath. Collapsing the flared distal end may crimp at least a portion of a prosthetic segment disposed thereunder to a reduced diameter onto the delivery catheter. Retracting within the catheter may comprise moving the remaining prosthetic segments into the sheath. Retracting may also comprise retracting a segment mover that is releasably coupled to the remaining prosthetic segments. Retracting may comprise retracting the outer sheath. The outer sheath may have a flared distal section that is engaged with at least one of the unselected prosthetic segments.

Moving the remaining prosthetic segments may comprise crimping a portion thereof back to a substantially unexpanded configuration, and the method may further comprise advancing the remaining prosthetic segments toward a distal end of the delivery catheter. Advancing may also include pushing the remaining prosthetic segments distally with a segment mover connected thereto. The method may also include delivering a therapeutic agent such as a restenosis inhibitor, to the treatment site. The therapeutic agent may be coupled to the prosthetic segments and it can be released therefrom.

In still another aspect of the present invention, a method for deploying a prosthesis into a treatment site in a body lumen comprises advancing a delivery catheter to the treatment site. The delivery catheter has a plurality of radially expandable prosthetic segments disposed thereon and they are at least partially covered by a sheath. The plurality of radially expandable prosthetic segments are also releasably interlocked with one another while unexpanded. Selecting a number of prosthetic segments to deploy into the body lumen allows the selected number of prosthetic segments to have a length that substantially traverses a length of a lesion at the treatment site and the selected number is less than the total number of prosthetic segments on the catheter. The method also includes partially expanding the selected number of prosthetic segments, wherein the selected number of prosthetic segments decouple from at least one other of the prosthetic segments which remain at least partially unexpanded on the delivery catheter during expansion of the selected number of prosthetic segments. The at least one other partially unexpanded prosthetic segment is retracted within the catheter so that it moves away from the selected number of expanded prosthetic segments and the selected number of prosthetic segments are then radially expanded into the treatment site.

Often the prosthetic segments are expanded by expanding an expansion member on the delivery catheter and the at least one other partially unexpanded prosthetic segment may be retracted after the expansion member is expanded. Sometimes a middle prosthetic segment is disposed between the selected number of prosthetic segments to be expanded and the at least one other prosthetic segment. The middle prosthetic segment may have a proximal portion which is unexpanded and a distal portion which is partially expanded when the selected number of prosthetic segments are expanded. A distal end of the middle prosthetic segment may be released from the selected number of prosthetic segments upon expansion thereof and a proximal end of the middle prosthetic segment may remain connected to the at least one other prosthetic segment which remains partially unexpanded.

The step of retracting may comprise retracting the middle prosthetic segment into the sheath and the sheath crimps the distal portion of the middle segment into an unexpanded shape. Retracting may also comprise retracting any unselected prosthetic segments into the sheath. Retracting the unselected prosthetic segments into the sheath may also crimp at least a portion of one of the segments into a substantially unexpanded configuration. The method may further comprise advancing the selected number of prosthetic segments toward a distal end of the delivery catheter prior to radial expansion thereof. Sometimes selecting may comprise advancing the selected number of prosthetic segments over an expandable member adjacent to a distal end of the delivery catheter. Selecting may also comprise positioning an expandable member that is coupled to a distal end of the delivery catheter such that the selected number of prosthetic segments are postioned thereover. Positioning may include retracting the expandable member into the selected number of prosthetic segments. Selecting may comprise actuating a control mechanism adjacent to a proximal end of the delivery catheter.

Partially expanding may comprise advancing the selected number of prosthetic segments over a ramped section of the delivery catheter while retracting any unselected prosthetic segments within the catheter may comprise crimping at least one of the unselected prosthetic segments to a reduced profile. The method may further comprise advancing the outer sheath distally so as to engage and move at least one of the selected number of prosthetic segments distally. Sometimes radially expanding the selected number of prosthetic segments comprises inflating a balloon disposed near a distal end of the delivery catheter. Radially expanding the selected number of prosthetic segments comprises flaring a distal end of the outer sheath, the distal end expanding with the selected prosthetic segments. Radially expanding the selected number of prosthetic segments may also comprise axially compressing a distal portion of the outer sheath as an expandable member expands. The method may further comprise collapsing a flared distal portion of the outer sheath. Radially expanding the selected number of prosthetic segments may comprise axially compressing a distal portion of the outer sheath as the expandable member expands. Additionally, the method may further include collapsing a flared distal portion of the outer sheath. Collapsing the flared portion crimps at least one prosthetic segment to a substantially unexpanded configuration having reduced profile onto the delivery catheter.

Sometimes the method may also include retracting an expandable member into the outer sheath. The method may further comprise delivering a therapeutic agent to the lesion, the therapeutic agent being coupled to the prosthetic segments and adapted to being released therefrom. The therapeutic agent may inhibit restenosis. The method may also comprise contracting an expandable member so that it may be withdrawn from the selected prosthetic segments into the outer sheath.

These and other embodiments are described in further detail in the following description related to the appended drawing figures.

### BRIEF DESCRIPTION OF THE DRAWINGS

FIG. 1 is a perspective view of a stent delivery catheter according to an exemplary embodiment of the present invention.

FIG. 2 shows a cross-sectional side view of the embodiment of FIG. 1.

FIG. 3 shows a perspective view of an alternative embodiment of a stent delivery catheter.

FIG. 4 shows the proximal and distal ends of the embodiment illustrated in FIG. 3.

FIGS. 5A-5H show the exemplary use of a stent delivery catheter to deliver a prosthesis.

FIGS. 6A-6H show another exemplary use of a stent delivery catheter to deliver a prosthesis.

FIG. 7 illustrates a radially expandable prosthetic segment in the unexpanded configuration.

FIG. 8 illustrates the prosthetic segment in FIG. 7 after it has been expanded.

FIG. 9 shows two unexpanded prosthetic segments releasably interlocked with one another.

FIG. 10 shows the two prosthetic segments in FIG. 9 after they have been expanded.

FIG. 11 shows two unexpanded prosthetic segments releasably interlocked with one another.

FIG. 12 shows the two prosthetic segments of FIG. 11 after they have been expanded.

FIG. 13 shows a system for automatically retracting prosthetic segments during balloon deflation.

FIG. 14 shows a system for automatically retracting prosthetic segments during balloon inflation.

FIG. 15 illustrates an alternative expansion member.

FIGS. 16A-16G illustrate several embodiments of flaring sheath distal ends.

FIGS. 17A-17B illustrates how an additional crimping member may recrimp any partially expanded stents.

FIGS. 18A-18B show how a flange may be used to help retract prosthetic segments.

### DETAILED DESCRIPTION OF THE INVENTION

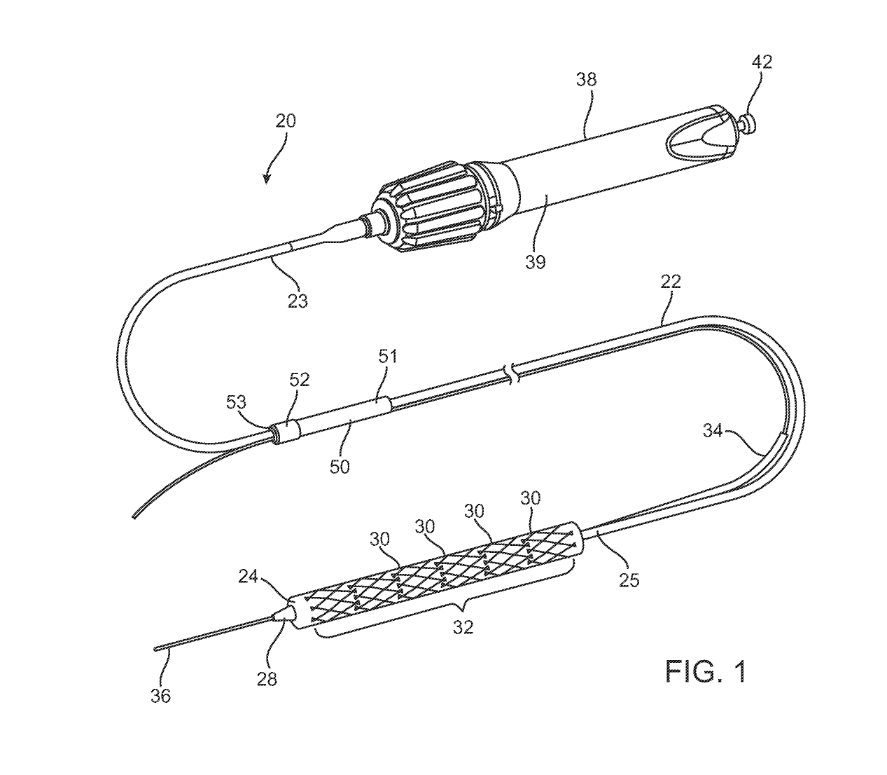

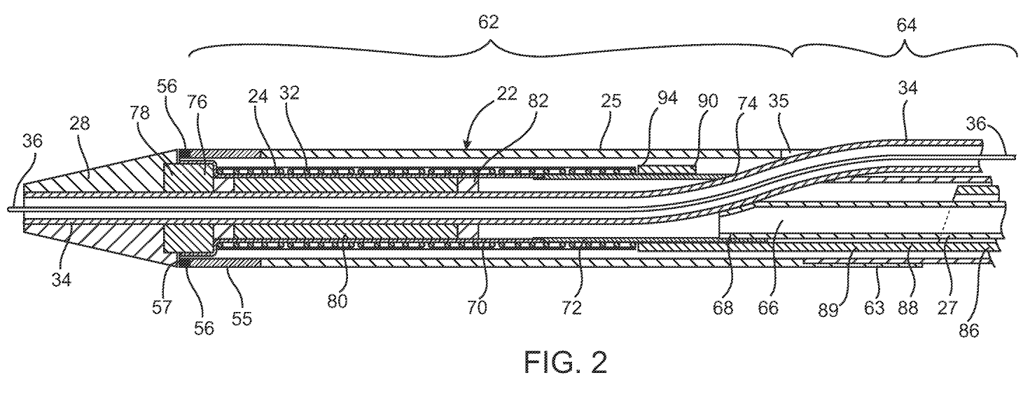

A first embodiment of a stent delivery catheter according to the present invention is illustrated in FIG. 1. Stent delivery catheter 20 includes a catheter body 22 comprising sheath 25 slidably disposed over an inner shaft 27 (seen in FIG. 2). An expandable member 24, preferably an inflatable balloon (shown in an inflated configuration), is mounted to inner shaft 27 and is exposed by retracting sheath 25 relative to inner shaft 27. A tapered nose cone 28, composed of a soft elastomeric material to reduce trauma to the vessel during advancement of the device, is mounted distally of expandable member 24. A stent 32, which preferably comprises a plurality of separate or separable stent segments 30 interleaving and releasably interlocked with one another, is disposed on expandable member 24 for expansion therewith. A guidewire tube 34 is slidably positioned through a guidewire tube exit port 35 in sheath 25 proximal to expandable member 24. A guidewire 36 is positioned slidably through guidewire tube 34, expandable member 24, and nose cone 28 and extends distally thereof.

A handle 38 is attached to a proximal end 23 of the sheath 25. The handle 38 performs several functions, including operating and controlling the catheter body 22 and the components included in the catheter body. Various embodiments of a preferred handle and additional details concerning its structure and operation are described in co-pending pending U.S. Patent Publication 2006/0282150 entitled “Devices and Methods for Operating and Controlling Interventional Apparatus,” the contents of which are incorporated herein by reference. Embodiments of other preferred handles and details concerning their structure and operation are described in co-pending U.S. patent application Ser. No. 10/746,466 filed Dec. 23, 2003 (Attorney Docket No. 021629-002200US), entitled “Devices and Methods for Controlling and Indicating the Length of an Interventional Element,” and U.S. patent application Ser. No. 11/614,271 filed Dec. 21, 2006 (Attorney Docket No. 021629-002210US), entitled “Automated Control Mechanisms and Methods for Custom Length Stent Apparatus,” the entire contents of all of the above referenced applications are hereby incorporated herein by reference.

The handle 38 includes a housing 39 that encloses the internal components of the handle. The inner shaft 27 is preferably fixed to the handle, while the outer sheath 25 is able to be retracted and advanced relative to the handle 38. An adaptor 42 is attached to the handle 38 at its proximal end, and is fluidly coupled to the inner shaft 27 in the interior of the housing of the handle 38. The adaptor 42 is configured to be fluidly coupled to an inflation device, which may be any commercially available balloon inflation device such as those sold under the trade name “Indeflator™”, available from Abbott (formerly Guidant Corporation of Santa Clara, Calif.). The adaptor 42 is in fluid communication with the expandable member 24 via an inflation lumen in the inner shaft 27 to enable inflation of the expandable member 24.

The outer sheath 25 and guidewire 36 each extend through a slider assembly 50 located on the catheter body 22 at a point between its proximal and distal ends. The slider assembly 50 is adapted for insertion into and sealing within a hemostatic valve, such as on an introducer sheath or guiding catheter, while allowing relative movement of the outer sheath 25 relative to slider assembly 50. The slider assembly 50 includes a slider tube 51, a slider body 52, and a slider cap 53. The slider assembly is described in further detail in U.S. Patent Publication 2007/0027521, filed Jun. 1, 2006 and entitled “Apparatus and Methods for Deployment of Multiple Custom-Length Prostheses,” (Attorney Docket No. XTNTNZ00700), the entire contents of which are incorporated herein by reference.

FIG. 2, shows a distal portion of the stent delivery catheter 20 in cross-section, where it may be seen that sheath 25 may be extended up to nose cone 28 to fully surround expandable member 24 and stent segments 32. A resilient section outer sheath 25 forms a garage 55 that is attached to the distal end 57 of outer sheath 25. This section is resilient and can flex with the stents and expandable member 24 as they expand and also this section collapses substantially back to its unexpanded state when the expandable member is collapsed. The garage 55 is a generally cylindrical member and preferably has a length at least as long as one of the stent segments 30 carried by the catheter, but preferably less than the combined length of two such stent segments. Additional details of garage 55 are described later in this application as well as in U.S. Patent Publication 2007/0027521, the entire contents of which are incorporated herein by reference. A radiopaque marker 56 is preferably formed integrally with or attached to the distal end of the garage 55 to facilitate visualization of the position of the sheath 25 using fluoroscopy.

As thus described, the sheath 25 has a distal extremity 62 configured to surround expandable member 24 and stent segments 32 disposed thereon when in an unexpanded configuration. Distal extremity 62 extends proximally to a junction 63, preferably aligned with the location of guidewire tube exit port 35, where distal extremity 62 is joined to a proximal extremity 64 that extends proximally to handle 38 (see FIG. 1). In a preferred embodiment, distal extremity 62 has a length of about 15-35 cm and proximal extremity 64 as a length of about 100-125 cm. Proximal extremity 64 may be constructed of a variety of biocompatible polymers, metals, or polymer/metal composites, preferably being stainless steel or Nitinol. Distal extremity 62 may be a polymer such as PTFE, FEP, polyimide, nylon, or Pebax, or combinations of any of these materials. In a preferred form, the distal extremity 62 comprises a composite of nylon, PTFE, and polyimide. The distal extremity is preferably reinforced with a metallic or polymeric braid to resist radial expansion when expandable member 24 is expanded. Sheath 25 may further have a liner surrounding its interior of low friction material such as PTFE to facilitate relative motion of sheath 25, stent segments 30, and pusher tube 86.

Preferably, proximal extremity 64 has a smaller transverse dimension than distal extremity 62 to accommodate the added width of guidewire tube 34 within the vessel lumen, as well as to maximize flexibility and minimize profile. In one embodiment, distal extremity 62 is a tubular member having a first outer diameter, preferably about 1.0-1.5 mm, and proximal extremity 64 is a tubular member having a second, smaller outer diameter, preferably about 0.7-1.0 mm.

Guidewire tube 34 is slidably positioned through guidewire tube exit port 35. The guidewire tube exit port 35 may be configured to provide a total or partial fluid seal around the periphery of guidewire tube 34 to limit blood flow into the interior of sheath 25 and to limit leakage of saline (or other flushing fluid) out of sheath 25. This may be accomplished by sizing guidewire tube exit port 35 appropriately so as to form a fairly tight frictional seal around guidewire tube 34 while still allowing the sliding motion thereof relative to sheath 25. Alternatively an annular sealing ring may be mounted in guidewire tube exit port 35 to provide the desired seal. Preferably, however, the guidewire tube exit port 35 is not totally fluid sealed, so as to provide a slight leakage or fluid flow to provide the ability to flush the distal extremity 62 of the catheter.

Guidewire tube exit port 35 will be positioned to provide optimal tracking of stent delivery catheter 20 through the vasculature and maximizing the ease with which the catheter can be inserted onto and removed from a guidewire to facilitate catheter exchanges. Usually, guidewire tube exit port 35 will be positioned at a location proximal to expandable member 24 when sheath 25 is extended fully distally up to nose cone 28, but a distance of no more than one-half the length of sheath 25 from distal end 57. In preferred embodiments for coronary applications, guidewire tube exit port 35 is spaced proximally a distance of about 20-35 cm from the distal end 57 of sheath 25.

Guidewire tube 34 should extend proximally from guidewire tube exit port 35 a distance at least as long as the longest possible stent that may be deployed, e.g., 30-200 mm depending upon the application, to allow for retraction of sheath 25 that distance while retaining a portion of guidewire tube 34 external to sheath 25. Preferably the guidewire tube 34 extends proximally a distance of about 35 to about 70 mm from the guidewire tube exit port 35 when sheath 25 is in a fully distal position, with the proximal end thereof disposed a distance of about 23-50 cm from the distal tip of nose cone 28. Where stent delivery catheter 20 is to be positioned through a guiding catheter, the proximal end of guidewire tube 34 will preferably be positioned so as to be within the guiding catheter when expandable member 24 is positioned at the target site for stent deployment. Guidewire tube 34 is preferably a highly flexible polymer such as PTFE, FEP, polyimide, or Pebax, and may optionally have a metal or polymer braid or fiber embedded in it to increase kink-resistance and tensile strength.

Inner shaft 27 forms an inflation lumen 66 that is in communication with the interior of expandable member 24. The inner shaft 27 may be formed of a polymer material such as PTFE, FEP, polyimide, or Pebax, or the inner shaft 27 may be a metal such as stainless steel or Nitinol.

Expandable member 24 has an expandable balloon member 70 that is joined to a non-expandable tubular leg 72. Expandable balloon member 70 is a semi-compliant polymer such as Pebax, polyurethane, or Nylon. Non-compliant, fully elastic, or other materials such as PTFE may also be used. Preferably, the compliance of the balloon member allows the expanded diameter of balloon member 70 to be adjusted by selecting the appropriate inflation pressure delivered thereto, thereby allowing customization of the deployed diameter of stent segments 30. For example, in one embodiment, balloon member 70 may be inflated to a pressure of between about 5 and about 12 atmospheres, allowing the deployed stent diameter to be adjusted from about 2.0 mm to 4.0 mm. Of course, larger and smaller stent diameters are also possible by utilizing appropriate stent geometry and applying suitable inflation pressures. Tubular leg 72 is preferably a polymer such as polyimide, PTFE, FEP, polyurethane, or Pebax and may optionally be reinforced with a metal or polymer braid or metal or polymer fibers. Tubular leg 72 has an open proximal end 74 through which guidewire tube 34 extends. Proximal end 74 of tubular leg 72 is fixed to distal end 68 of inner shaft 27 and to guidewire tube 34, forming a fluid-tight seal. Guidewire tube 34 passes through the interior of balloon member 70 and is mounted to nose cone 28, thereby providing a passage through the distal portion of catheter body 22 through which guidewire 36 may pass. Balloon member 70 has a distal end 76 that extends over an annular stop 78, which is mounted to the distal end of guidewire tube 34 and/or nose cone 28. Distal end 76 of balloon member 70 may be bonded to stop 78, guidewire tube 34, and/or nose cone 28. The stop 78 has a size and shape selected to engage stent segment 32 and provide a stop against which stent segments 32 can be located in the ideal deployment position without being pushed beyond the distal end of balloon member 70. Additional details concerning stent stops suitable for use in the devices and methods described herein are disclosed in U.S. Pat. No. 7,182,779 (Attorney Docket No. 021629-000360), which is hereby incorporated by reference.

Preferably, the stop 78 has a partial cylindrical shape, rather than a full cylindrical shape, as a relief to reduce interference with garage 55. The stop 78 limits distal movement of the stent segments 32, while reducing interference between stop 78 and the interior of garage 55.

Optionally, within the interior of balloon member 70 an annular base member 80 is mounted to guidewire tube 34 and has a diameter selected to urge balloon member 70 against stent segments 30 in their unexpanded configuration, thereby providing frictional engagement with stent segments 30. This helps to limit unintended sliding movement of stent segments 30 on balloon member 70. Base member 80 may be made of a soft elastomer, foam, or other compressible material.

Optional annular radiopaque markers 82 may be mounted to the guidewire tube 34, facilitating visualization of the location of balloon member 70 with fluoroscopy and enabling appropriate positioning of stent segments 30 on balloon member 70. The radiopaque markers 82 are preferably located at regular intervals along the length of the guidewire tube 34. Such markers may be made of various radiopaque materials such as platinum/iridium, tantalum, gold, and other materials.

A pusher tube 86, also referred to as a prosthetic segment moving tube, or segment mover, is slidably disposed over inner shaft 27. The pusher tube 86 contains three primary sections, a distal extension 88, a ribbon portion 89, and a proximal portion 90. The proximal portion 90 extends from the handle 38 over the inner shaft 27 and to the ribbon portion 89. The proximal portion 90 is preferably formed of a tubular material to provide high column strength but adequate flexibility to extend through the vasculature from an access site to the coronary ostia or other target vascular region. A preferred material is stainless steel hypotube. The ribbon portion 89 of the pusher tube corresponds with the location of the guidewire exit port 35 on the outer sheath 25. The ribbon portion 89 is formed of a partial-tube, in order to provide an opening to allow the guidewire tube 34 to pass through to the exit port 35. The proximal portion of the ribbon portion 89 is formed out of the same tubular material that makes up the proximal portion 90 of the pusher tube, e.g., stainless steel hypotube. The proximal portion of the ribbon portion 89 is joined to the distal portion of the ribbon 89, such as by a weld or the ribbon portion and proximal portion may be formed from the same hypotube which is laser cut in the appropriate geometry. The distal extension 88 is preferably formed of a slotted tube of rigid material, such as stainless steel or Nitinol in order to make the pusher tube more flexible so as to be capable of bending around a transverse axis. Tip 94 of pusher tube 86 preferably has a geometry with axial projections similar to or complementary to those of stent segments 32 so as to releasably interlock therewith.

Pusher tube 86 extends longitudinally within the outer sheath 25 and over the inner shaft 27 through most of the length of the catheter body 22. The distal extension 88 is slidable over the tubular leg 72 and engages the stent segment 32 at the proximal end of the line of stent segments 32. At its proximal end (not shown), the pusher tube 86 is coupled to an actuator associated with the handle 38 (see FIG. 1). In this way, the pusher tube 86 can be moved relative to inner shaft 27 to urge the stent segments 32 proximally or distally over the expandable member 24 until they engage the stop 78. The distal end of pusher tube 86 is often releasably coupled with an end of the proximal-most stent, thereby facilitating the ability of the pusher tube 86 to move the stent segments 32 both proximally as well as distally.

It can be seen that with sheath 25 retracted a desired distance, expandable member 24 is allowed to expand when inflation fluid is delivered through inflation lumen 66, thereby expanding a desired number of stent segments 32 exposed distally of sheath 25. The remaining portion of expandable member 24 and the remaining stent segments 32 within sheath 25 are constrained from expansion by sheath 25. Additional details about the delivery catheter are disclosed in U.S. Patent Publication 2007/0027521, the entire contents of which have previously been incorporated herein by reference.

Stent segments 30 are slidably positioned over balloon member 70 and releasably interlocked with one another. Depending upon the number of stent segments 32 loaded in stent delivery catheter 20, stent segments 30 may be positioned over both balloon member 70 and tubular leg 72. In an exemplary embodiment, each stent segment is about 2-20 mm in length, more preferably 2-8 mm in length, and 3-50 stent segments may be positioned end-to-end in a line over balloon member 70 and tubular leg 72.

Stent segments 30 are preferably a malleable metal so as to be plastically deformable by expandable member 24 as they are expanded to the desired diameter in the vessel. Stent segments 30 may also be composed of polymers or other suitable biocompatible materials including bioabsorbable or bioerodable materials.

In preferred embodiments, stent segments 30 are coated with a drug that inhibits restenosis, such as Rapamycin, Paclitaxel, Biolimus A9 (available from BioSensors International), analogs, prodrugs, or derivatives of the foregoing, or other suitable agent, preferably carried in a durable or bioerodable polymeric or other suitable carrier material. Alternatively, stent segments 30 may be coated with other types of drugs and therapeutic materials such as antibiotics, thrombolytics, anti-thrombotics, anti-inflammatories, cytotoxic agents, antiproliferative agents, vasodilators, gene therapy agents, radioactive agents, immunosuppressants, and chemotherapeutics. Several preferred therapeutic materials are described in U.S. Patent Publication No. 2005/0038505, entitled “Drug-Delivery Endovascular Stent and Method of Forming the Same,” filed Sep. 20, 2004, which is incorporated herein by reference. Such materials may be coated over all or a portion of the surface of stent segments 30, or stent segments 30 may include apertures, holes, channels, pores, or other features in which such materials may be deposited. Methods for coating stent segments 32 are described in the foregoing published patent application. Various other coating methods known in the art may also be used, including syringe application, spraying, dipping, inkjet printing-type technology, and the like.

Stent segments 30 may have a variety of configurations, including those described in copending application Ser. No. 10/738,666, filed Dec. 16, 2003 (Attorney Docket No. 21629-000510), which is incorporated herein by reference. Other preferred stent configurations are described below. Stent segments 30 are preferably releasably interlocked with one another, although it is feasible that stent segments 30 could also be separate from one another or stent segments could be coupled together with a frangible connector such as those disclosed in U.S. patent application Ser. No. 10/306,813, filed Nov. 27, 2002 (Attorney Docket No. 21629-000320), the entire contents of which is incorporated herein by reference.

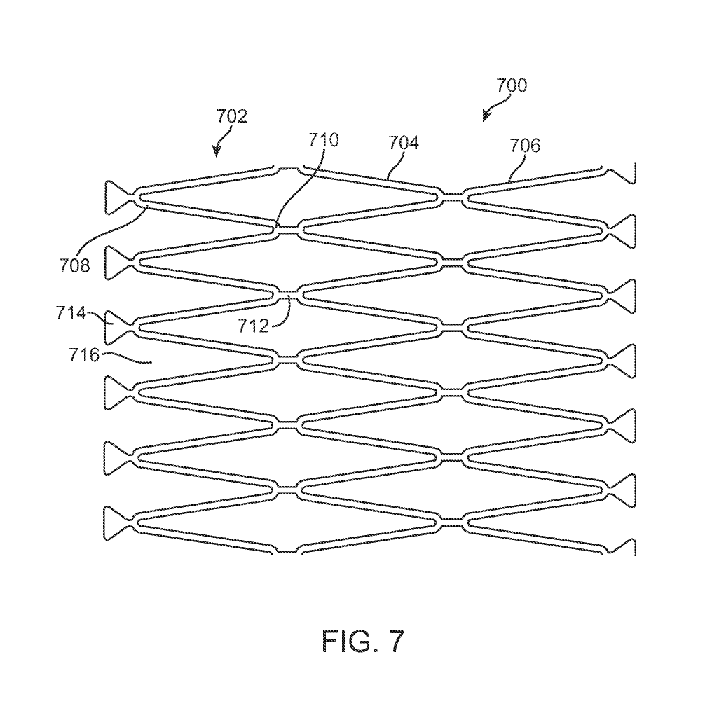

FIG. 7 illustrates a preferred embodiment of a stent segment geometry 700. In FIG. 7, stent segment 700 is shown unrolled and flattened for ease in viewing. Stent segment 700 made up of three parallel columns 702, 704, 706, with each column comprising a sine wave like pattern of elongate struts 708 coupled together with a U-shaped connector 710. The columns of sine waves 702, 704, 706 are connected together with a bridge 712. Additionally, both ends of stent segment 700 have enlarged triangular heads 714. The space 716 between triangular heads 714 forms a receptacle area that can releasably interlock with the enlarged triangular head 714 of an adjacent stent segment 700 in the unexpanded configuration, as seen in FIG. 1. FIG. 8 shows stent segment 700 in the expanded state and receptacle 716 enlarged so that adjacent stent segments 700 may be uncoupled from one another.

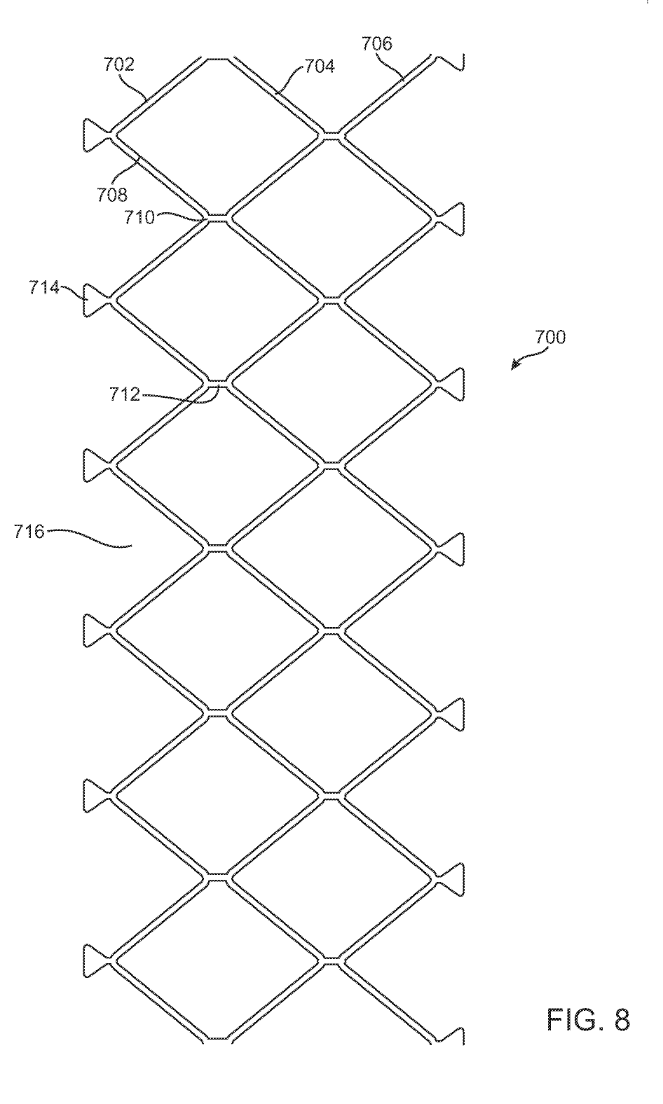

FIG. 9 illustrates another stent segment geometry that may be utilized. In FIG. 9, stent segments 900 are releasably coupled together. Again, stent segments 900 are shown in a planar shape for clarity. Stent segments 900 comprise parallel rows 922A, 922B and 922C of I-shaped cells 924 formed into a cylindrical shape around a central longitudinal axis. Cells 924 have upper and lower axial slots 926 and a connecting circumferential slot 928. Upper and lower slots 926 are bounded by upper axial struts 932, lower axial struts 930, curved outer ends 934, and curved inner ends 936. Circumferential slots 928 are bounded by outer circumferential strut 938 and inner circumferential strut 940. Each I-shaped cell 924 is connected to the adjacent I-shaped cell 924 in the same row 922A by a circumferential connecting strut 942. Row 922A is connected to row 922B by the merger or joining of curved inner ends 936 of at least one of upper and lower slots 926 in each cell 924. The ends of each stent segment 900 have enlarged triangular heads 950 that form a receptacle 952 which can releasably receive enlarged heads 950, thereby coupling segments 900 together.

In FIG. 9 the stent includes a bulge 944 in upper and lower axial struts 930, 932 extending circumferentially outwardly from axial slots 926. These give axial slots 926 an arrowhead or cross shape at their inner and outer ends. The bulge 944 in each upper axial strut 930 extends toward the bulge 944 in a lower axial strut 932 in the same cell 924 or in an adjacent cell 924, thus creating a concave abutment 946 in the space between each axial slot 926. Concave abutments 946 are configured to receive and engage the ends of an adjacent stent segment 900, thereby allowing interleaving of adjacent stent segment ends while maintaining spacing between the stent segments. The axial location of bulges 944 along upper and lower axial struts 930, 932 may be selected to provide the desired degree of inter-segment spacing.

FIG. 10 shows stent 930 of FIG. 9 in an expanded condition, again, unrolled and flattened out for clarity. It may be seen that axial slots 924 are deformed into a circumferentially widened modified diamond shape with bulges 944 on the now diagonal upper and lower axial struts 930, 932. Circumferential slots 928 are generally the same size and shape as in the unexpanded configuration. Bulges 944 have been pulled away from each other to some extent, but still provide a concave abutment 946 to maintain a minimum degree of spacing between adjacent stent segments. As in the earlier embodiment, some axial shortening of each segment occurs upon expansion and stent geometry can be optimized to provide the ideal intersegment spacing. Because receptacle 952 has expanded, the enlarged triangular head 950 of an adjacent stent segment is no longer captured therein and hence adjacent stent segments 900 may be released from one another.

It should also be noted that the embodiment of FIGS. 9-10 also enables access to vessel side branches blocked by stent segment 900. Should such side branch access be desired, a dilatation catheter may be inserted into circumferential slot 928 and expanded to provide an enlarged opening through which a side branch may be entered.

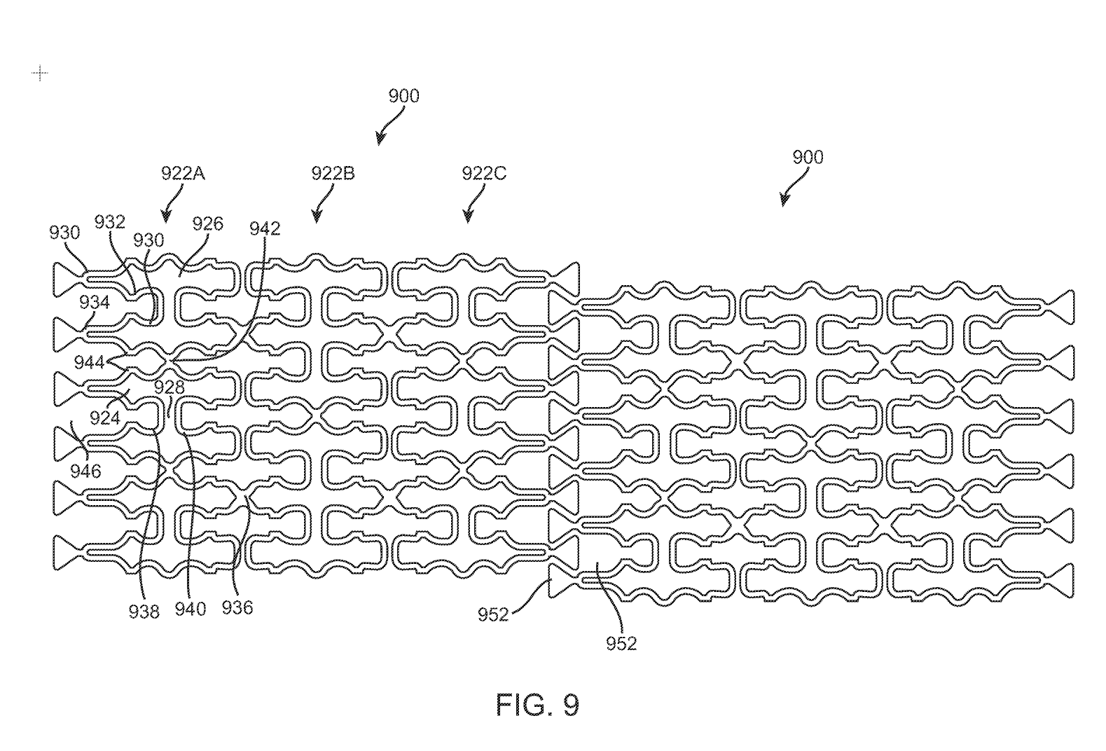

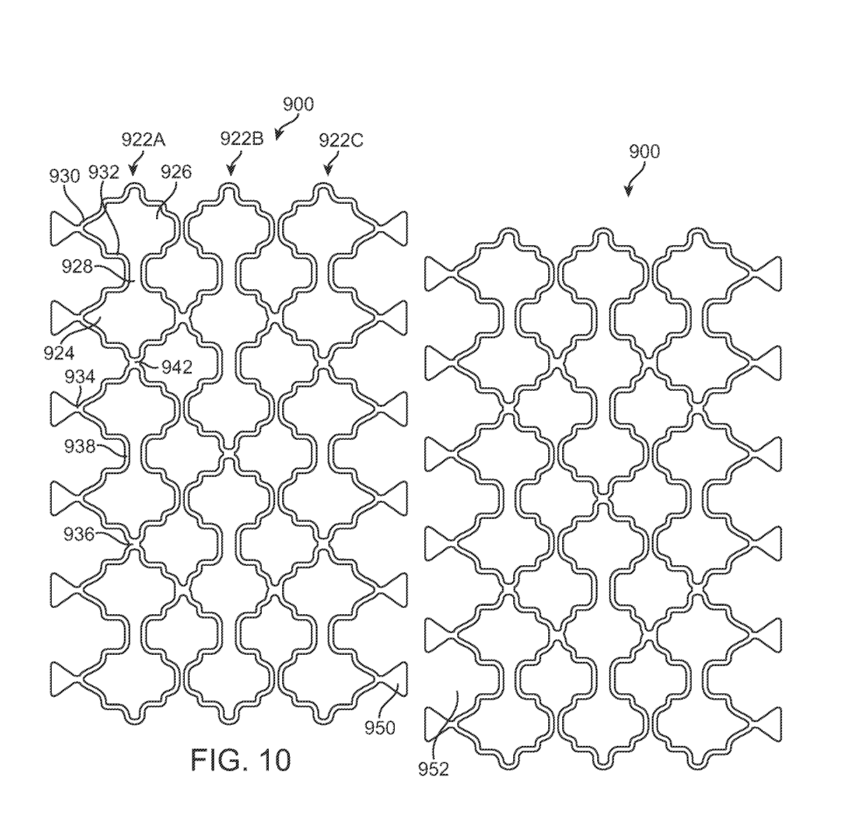

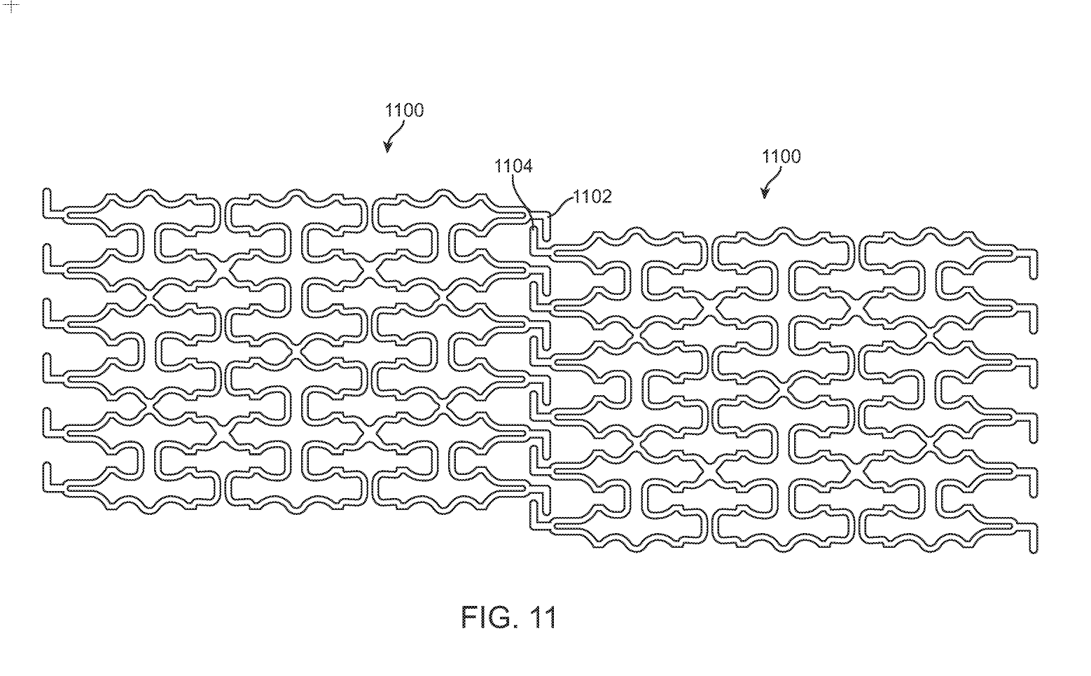

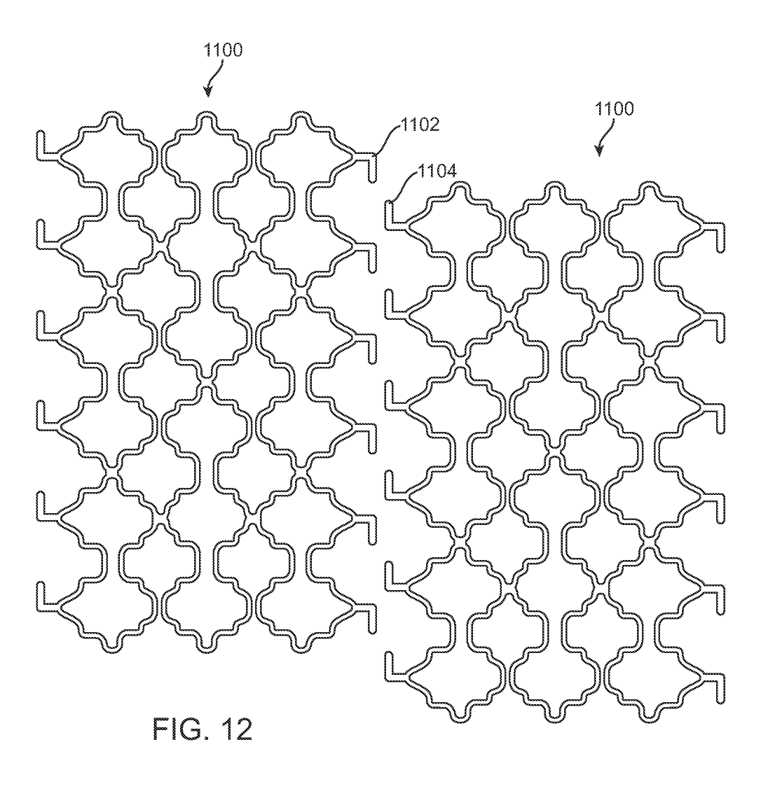

FIG. 11 shows a similar stent segment 1100 to that of FIGS. 9-10, with the major difference being that the enlarged triangular heads 950 have been replaced by L-shaped axially extending arms 1102, 1104. One side of stent segment 1100 has arms 1102 facing downward while the opposite end of stent segment 1100 has arms 1104 facing upward. This allows adjacent stent segments 1100 to releasably interlock with one another while the stents are unexpanded, and when stent segments expand as in FIG. 12, the arms are displaced away from one another so that they no longer interlock and thus the two stent segments may be disengaged and moved away from one another.

It should be recognized to one of ordinary skill in the art that many stent geometries may be used or modified to include interlocking tabs or arms so that adjacent stent segments may be releasably coupled together. Other examples of stent geometries that could be used in include those that are disclosed in U.S. Pat. Nos. 6,315,794; 5,980,552; 5,836,964; 5,527,354; 5,421,955; 4,886,062; and 4,776,337; the entire contents of which are incorporated herein by reference.

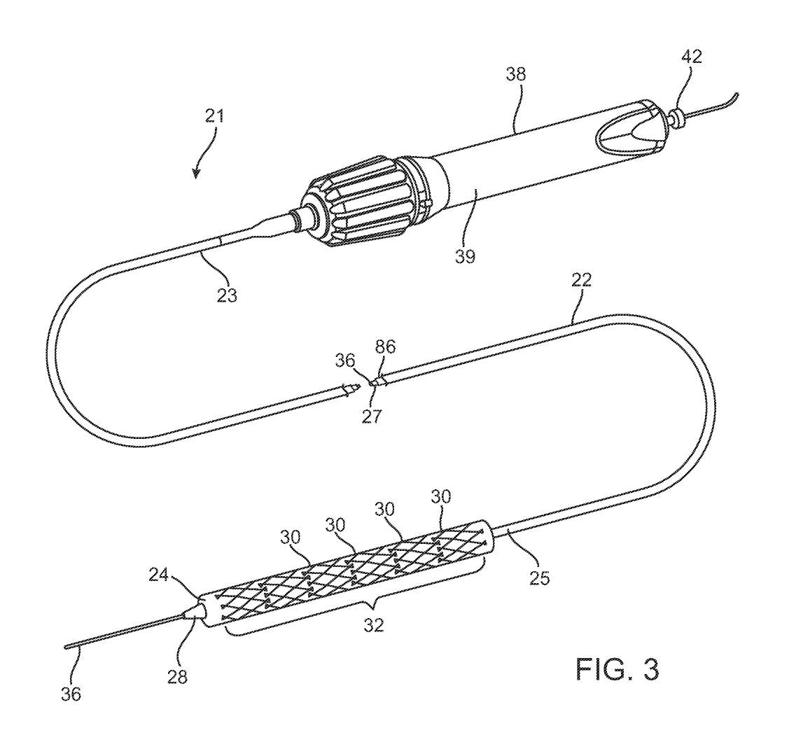

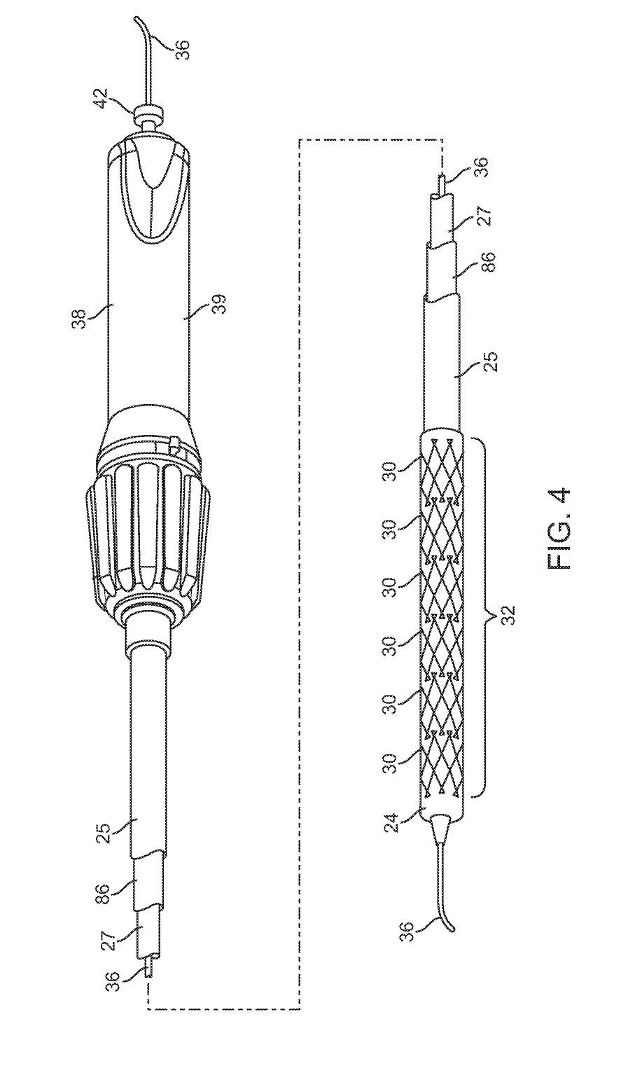

FIG. 3 shows an alternative embodiment of the stent delivery catheter 21. The major difference between the embodiment of FIG. 3 and that of FIG. 1 is that in FIG. 3, there is no guidewire tube. The embodiment of FIG. 3 is an over the wire stent delivery catheter having coaxial inner shaft 27, pusher tube 86 and outer sheath 25 as highlighted in FIG. 4. Other aspects of stent delivery catheter 21 such as handle 38, handle body 39, adapter 42 and stent 32 with stent segments 30, guidewire 36, nose cone 28 and balloon 24 generally take the same form as previously described with respect to the embodiment of FIG. 1. Over the wire stent delivery catheters provide some advantages that rapid exchange catheters do not, such as providing a more pushable catheter as well as allowing easy exchange of the catheter and/or guidewire, but over the wire systems generally require the use of a much longer guidewire which can be awkward to handle. FIG. 4 is an enlarged view of the over the wire delivery catheter seen in FIG. 3.

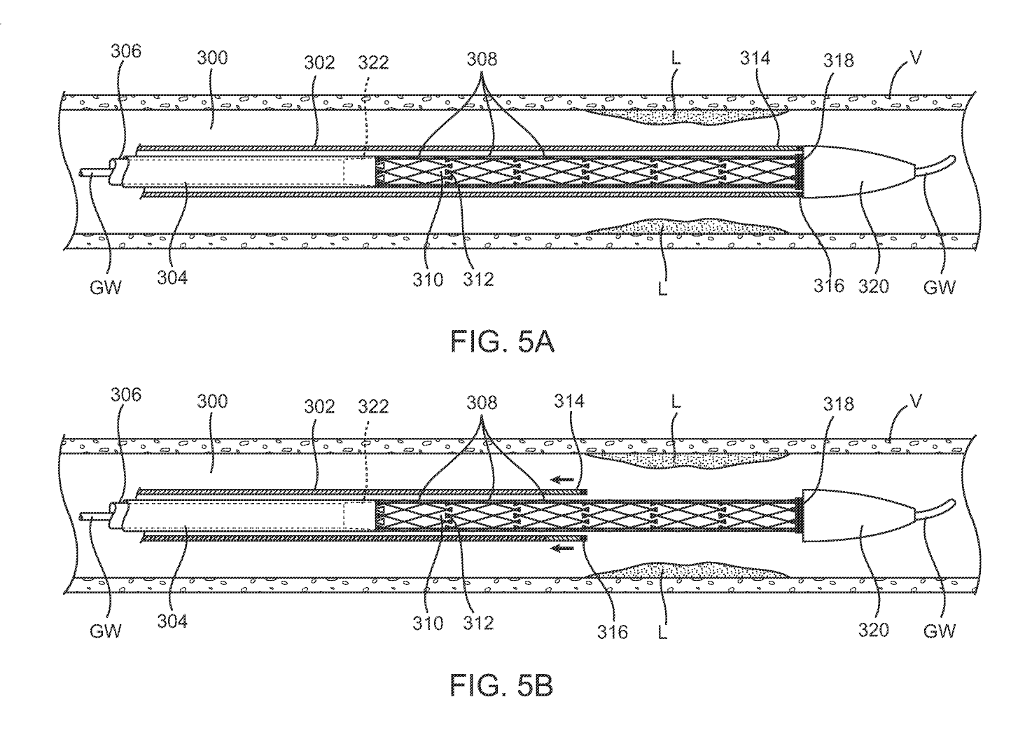

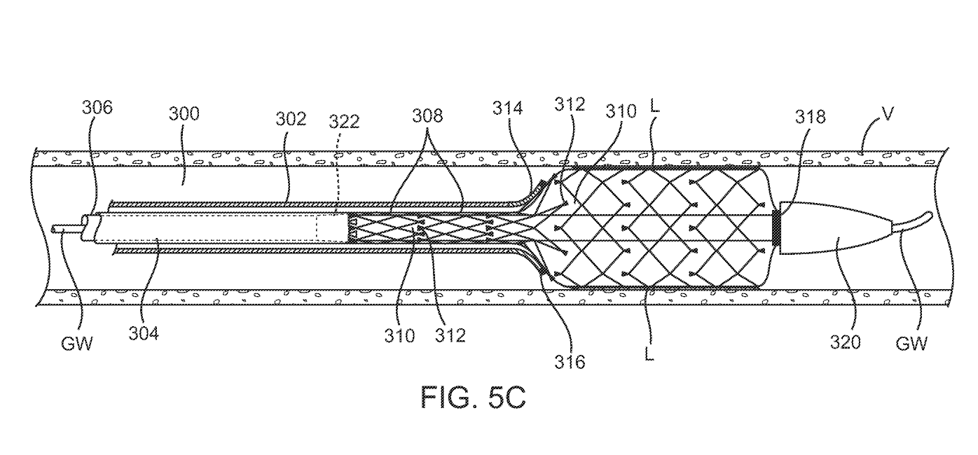

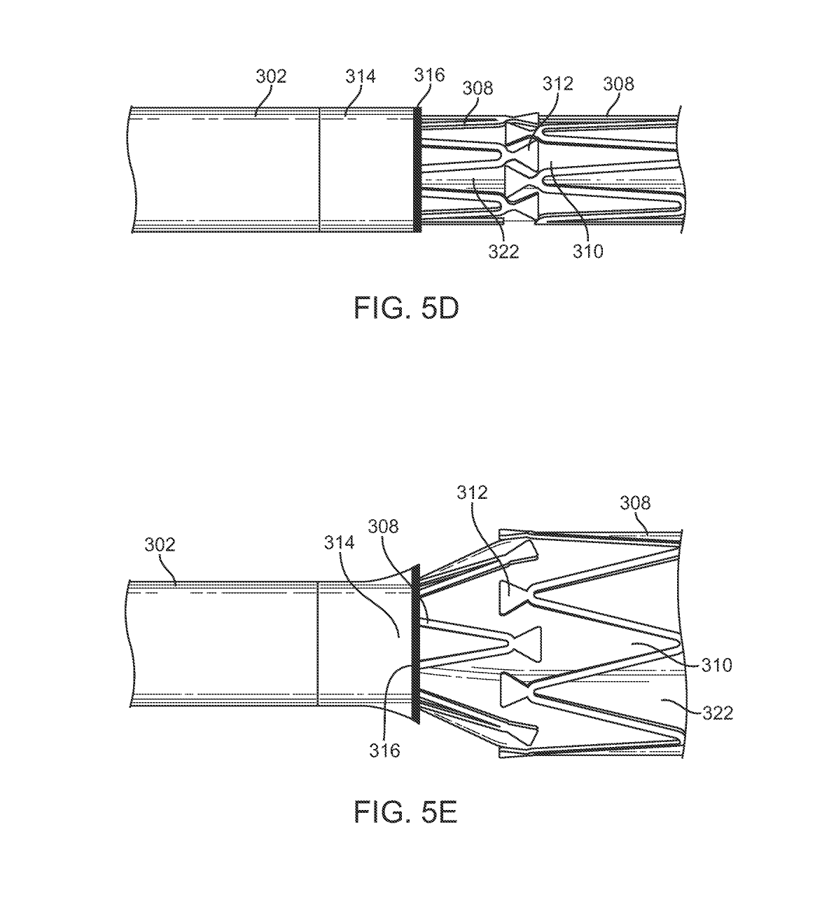

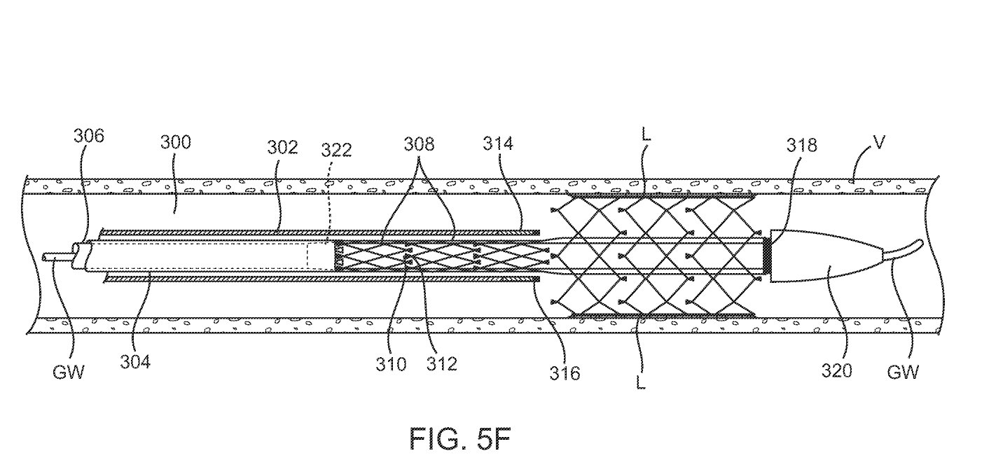

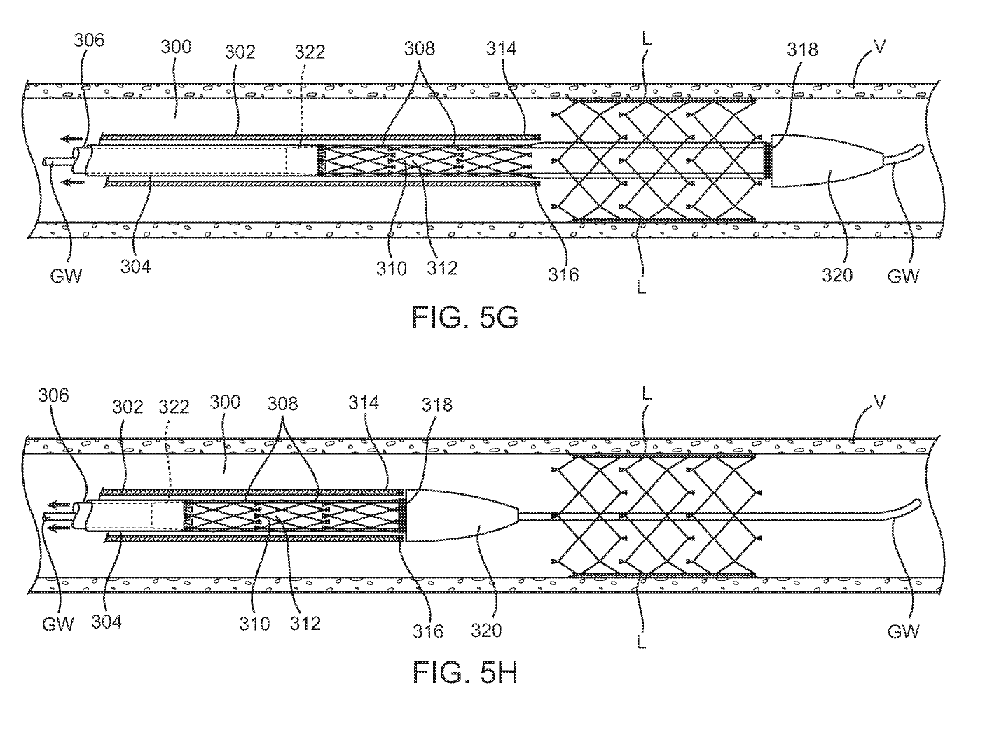

Referring now to FIGS. 5A-5H, the use of the stent delivery catheter illustrated in FIG. 1 will be described. While the invention will be described in the context of a coronary artery stent procedure, it should be understood that the invention is useful in any of a variety of blood vessels and other body lumens in which stents are deployed, including the carotid, femoral, iliac and other arteries, as well as veins and other fluid-carrying vessels such as the ureter, urethra and bile ducts. A guiding catheter (not shown) is first inserted into a peripheral artery such as the femoral artery and advanced to the ostium of the target coronary artery. A guidewire GW is then inserted through the guiding catheter into the coronary artery V where lesion L is to be treated. The proximal end of guidewire GW is then inserted through nose cone 320 and guidewire tube 34 outside the patient's body and stent delivery catheter 300 is slidably advanced over guidewire GW and through the guiding catheter into the coronary artery V. Slider assembly 50 is positioned within the hemostasis valve at the proximal end of the guiding catheter, which is then tightened to provide a hemostatic seal with the exterior of the slider body 52. Stent delivery catheter 300 is positioned through a lesion L to be treated such that nose cone 320 and stent stop/radiopaque marker 318 are distal to lesion L, as viewed under fluoroscopy or other imaging modality. During this positioning, sheath 302 is positioned distally up to nose cone 320 so as to surround expandable member 322 and all of the stent segments 308 thereon. Expandable member 322 here is a balloon coupled to inner shaft 306. The distal end of outer sheath 302 comprises a resilient region 314 that can flex with the stents 308 as they expand. Other embodiments of the resilient region 314 are discussed later in this disclosure. Additionally, a radiopaque marker 316 may be included near the tip of the resilient section 314 of outer sheath 302. Stent segments 308 have enlarged heads 312 that are adapted to releasably interlock with the region 310 between enlarged heads 312 on an adjacent stent segment. Pusher 304 also has enlarged heads extending from the pusher 304 in order to releasably interlock the pusher 304 with the proximal-most stent segment 308. Additional details on this will be described below.

In FIG. 5B, outer sheath 302 is retracted proximally so that radiopaque marker 316 is proximal to lesion L, thus the distance between radiopaque marker 316 and stent stop/radiopaque marker 318 represents the lesion length and the corresponding number of stent segments 308 required to traverse this length is then exposed. Here, three stent segments 308 are exposed from sheath 302 and a portion of a fourth stent segment 308 is partially disposed under outer sheath 302 and under the resilient distal tip 314 of sheath 302. During retraction of outer sheath 302, pusher 304 remains stationary and therefore stent segments 308 also remain stationary over balloon 322. The motion of the outer sheath 302 and pusher 304 is controlled by actuators on handle 38 in FIG. 1. FIG. 5D is an enlarged view of the stent segments 308 disposed over balloon 322 as the outer sheath 302 is retracted. Stent segments remain releasably interlocked with one another because enlarged head regions 312 are coupled with the space between enlarged head regions 310 on an adjacent stent segment 308.

In FIG. 5C, balloon 322 is radially expanded, thereby expanding the stent segments 308 exposed from sheath 302 into lesion L. The resilient section 314 of outer sheath 302 also expands and flares open with stents 308 and balloon 322, thereby partially expanding a portion of stent 308 disposed under resilient section 314 of sheath 302. The remaining portion of stent 308 does not expand since the stent 308 and balloon 322 is constrained by outer sheath 302 and therefore prevented from radially expanding. In some embodiments, a longer resilient section 314 of sheath 302 may be used in order to fully cover the partially expanded stent 308, thereby preventing stent struts from potentially piercing the vessel wall. Outer sheath 302 may be reinforced in order to help constrain balloon 322 from expanding. As stent segments 308 expand, the space 310 between enlarged head regions 312 also expand and therefore the enlarged heads 312 of stent segments 308 are no longer interlocked with an adjacent stent segment 308, and stent segments 308 may then be axially moved as described below. Enlarged head regions 321 may risk puncturing the proximal, tapered end of balloon 322 as balloon 322 is radially expanded. Therefore, in some embodiments, the tapered end of the balloon may be modified to prevent the occurrence of this puncturing. For example, a protective layer may be disposed on the proximal, tapered end of balloon 322, the thickness of the proximal, tapered end of balloon 322 may be greater than the main body of balloon 322, or the proximal, tapered end of balloon 322 may consist of a different material the main body of balloon 322. FIG. 5E illustrates how the resilient section of sheath 302 flares during balloon 322 expansion and also how adjacent stent segments unlock from one another.

In FIG. 5F, balloon 322 is deflated causing resilient section 314 to collapse back to its original shape, thereby crimping stent 308 that was partially expanded, and completely releasing the unexpanded stent segments from those that were expanded and left implanted at the lesion L. Because a portion of stent 308 remains exposed from resilient section 314, it may not be fully crimped back down over balloon 322 and thus in FIG. 5G, pusher tube 306 (also referred to as a prosthetic segment moving tube or segment mover) is retracted proximally. Pusher tube 304 is releasably coupled to the remaining stent segments 308 and therefore stent segments are drawn back into sheath 302, further crimping the partially expanded stent segment 308 down onto balloon 322. Alternatively, sheath 302 may be advanced distally to cover all of stents 308. As mentioned above, a longer resilient section 314 of sheath 302 may be used, in this case, to help crimp the partially expanded stent segment 308 back down onto balloon 322, which would potentially eliminate the additional step of retracting pusher 304 and stents 308 proximally into sheath 302. Additionally, in some embodiments, after the stents 308 have been constrained by sheath 302, the balloon may be reinflated in order to perform a post dilation of the lesion and implanted stents.

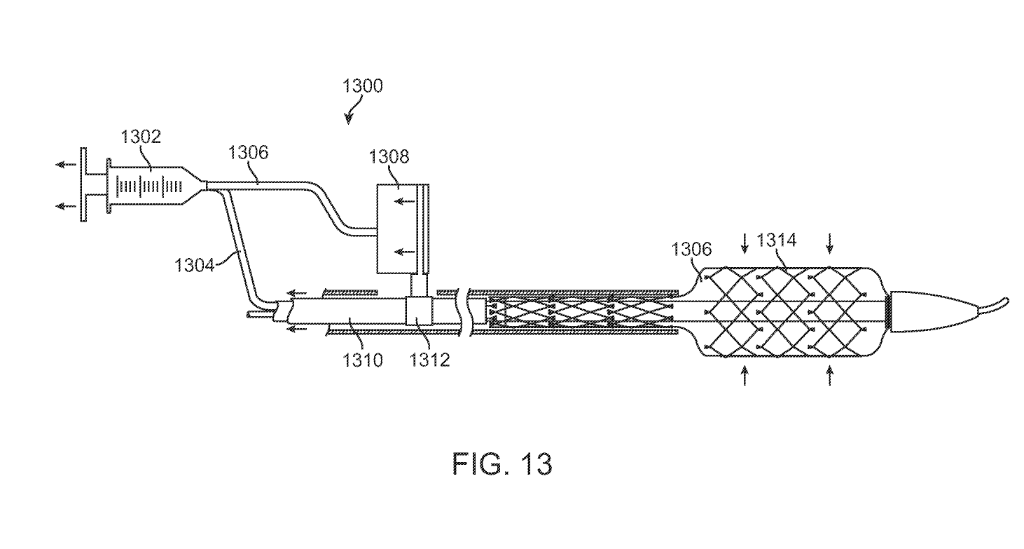

Some embodiments may also simultaneously deflate the balloon and retract the stents into the sheath. For example, in FIG. 13, as inflation device 1302 is retracted, fluid is withdrawn from balloon 1306 via fluid path 1304, deflating balloon 1306 leaving expanded stents 1314 deployed at a treatment site. As the inflation device 1302 is retracted, fluid is also withdrawn from piston 1308 via fluid path 1306, which simultaneously retracts pusher tube 1310 because it is coupled by coupling mechanism 1312 to piston 1308. Thus, as balloon 1306 is deflated, the unselected stents are simultaneously retracted into the outer sheath.

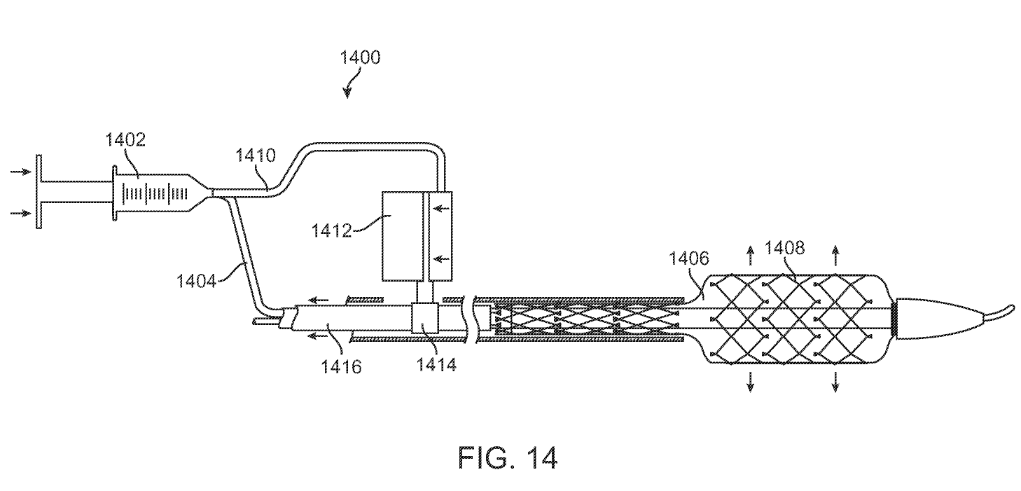

In still other embodiments, the unselected stents may be automatically withdrawn into the outer sheath as the balloon is expanded. For example, in FIG. 14, fluid is delivered from inflation device 1402 to balloon 1406 via fluid path 1404, thereby expanding the balloon 1406. Simultaneously, fluid is also delivered via path 1410 to piston 1412. Piston 1412 is coupled by coupling mechanism 1414 to pusher 1416 so that as balloon 1406 exapands, piston 1412 is retracted, thereby also retracting pusher 1416 which in turn retracts the unselected group of stent segments from those selected for delivery.

In FIG. 5H, inner shaft 306 is retracted while pusher 304 and outer sheath 302 remain stationary in order to advance all the remaining stent segments 308 distally until the distal most stent segment 308 is stopped by stent stop/radiopaque marker 318. Alternatively, pusher 306 may be advanced distally pushing the remaining stent segments distally. The delivery catheter 300 is now “reset” and may be removed from the treatment site and moved to another lesion site for stent delivery. It is possible that the stent resetting step may be combined with the step where a partially expanded stent is retracted into the sheath to recrimp it onto the balloon, thereby making the procedure less cumbersome.

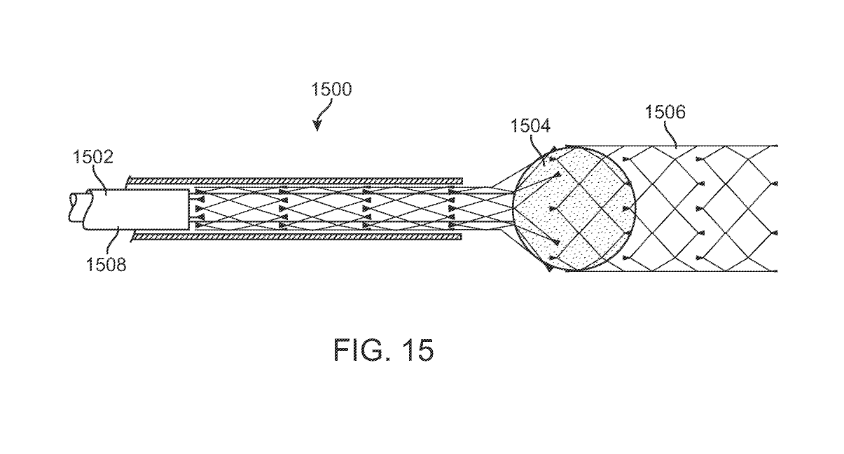

In the previous exemplary embodiment, an expandable balloon is used to expand the stent segments selected for deployment. Other expansion members may also be used. For example, as shown in FIG. 15, a spherical ball 1504 may be attached to the catheter shaft 1502. Thus, in catheter delivery system 1500, as ball 1504 is retracted into stents 1506, the ball 1504 deforms the stents and expands them into the desired configuration. Other geometries such as a cone could also be used to expand the stents into a treatment site. Additionally, the expansion member may be used to decouple adjacent stent segments followed by a separate step of balloon/stent segment expansion, rather than simultaneous decoupling and expansion.

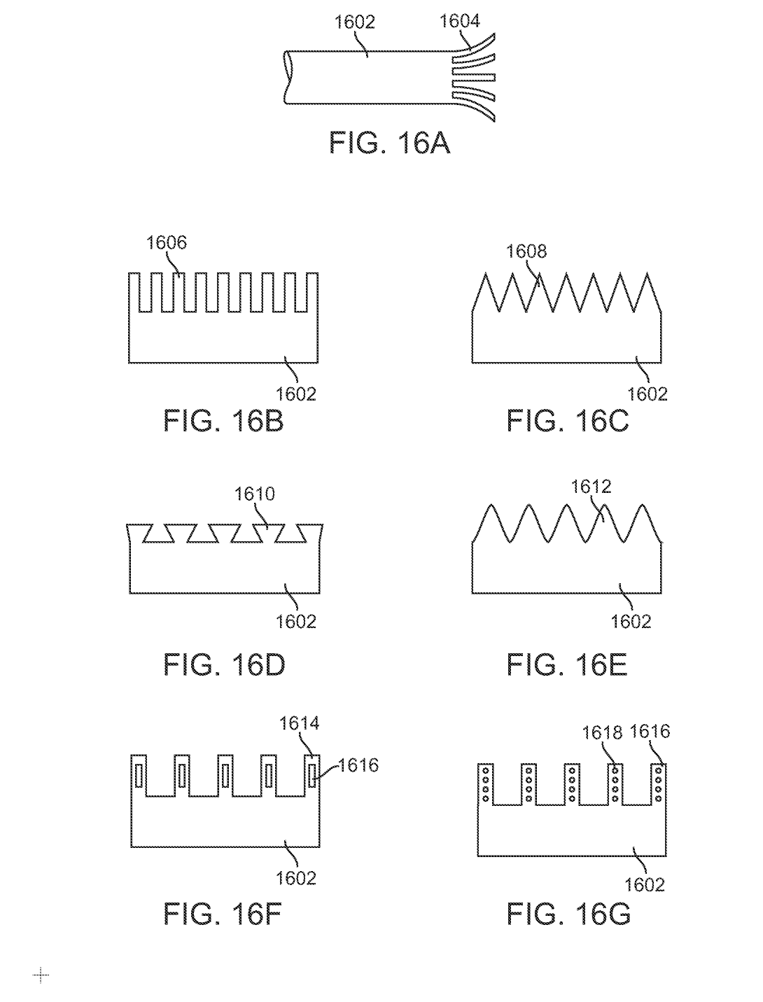

As previously mentioned in the embodiment above, the outer sheath may have a resilient distal section that flares open with the stent segments as they are expanded and then contracts back to it original shape when the balloon is deflated so as to help re-crimp the partially expanded stent back to its original diameter. In the embodiment above, the resilient section is an annular section coupled to the sheath. Other distal tip configurations may also be used such as those illustrated in FIGS. 16A-16G. For example, in FIG. 16A, a plurality of resilient fingers 1604 are coupled to outer sheath 1602. These fingers may be elongated and rectangular in shape such as fingers 1606 in FIG. 16B or they may be scalloped such as fingers 1608 in FIG. 16C. FIG. 16D shows triangular shaped fingers 1610 while FIG. 16E illustrates fingers formed form a wave-like pattern 1612. Rectangular fingers 1614 may also be hinged as in FIG. 16F in order to control their flexibility using rectangular slots 1616 or a series of apertures 1618 in the fingers 1616 of FIG. 16G.

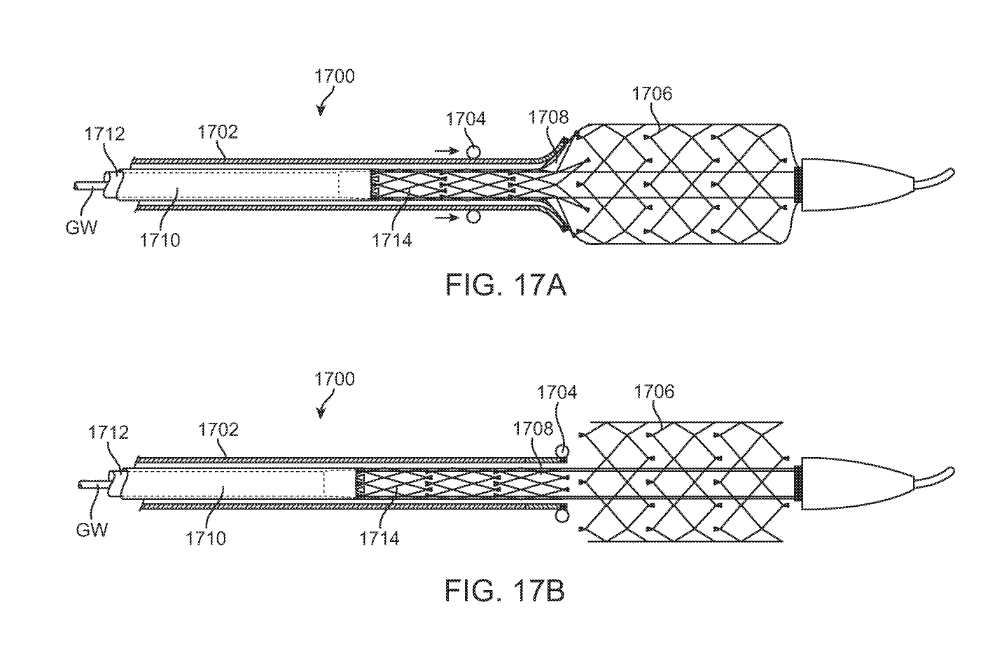

Other features which may be included in the previous embodiment include a crimping member 1704 as seen in FIG. 17A. After stents 1706 are expanded by a balloon, the balloon is then deflated. The resilient section of the outer sheath will help to re-crimp the partially expanded stent 1708. To ensure that the partially expanded stent 1708 is fully crimped, crimping member 1704 is advanced distally over the outer sheath thereby compressing the sheath and the partially expanded stent 1708 back down to their original diameters, as this is illustrated in FIG. 17B. Crimping member 1704 may be an o-ring which is slidably advanced over the outer sheath, or it may be an additional tube that slides over the outer sheath.

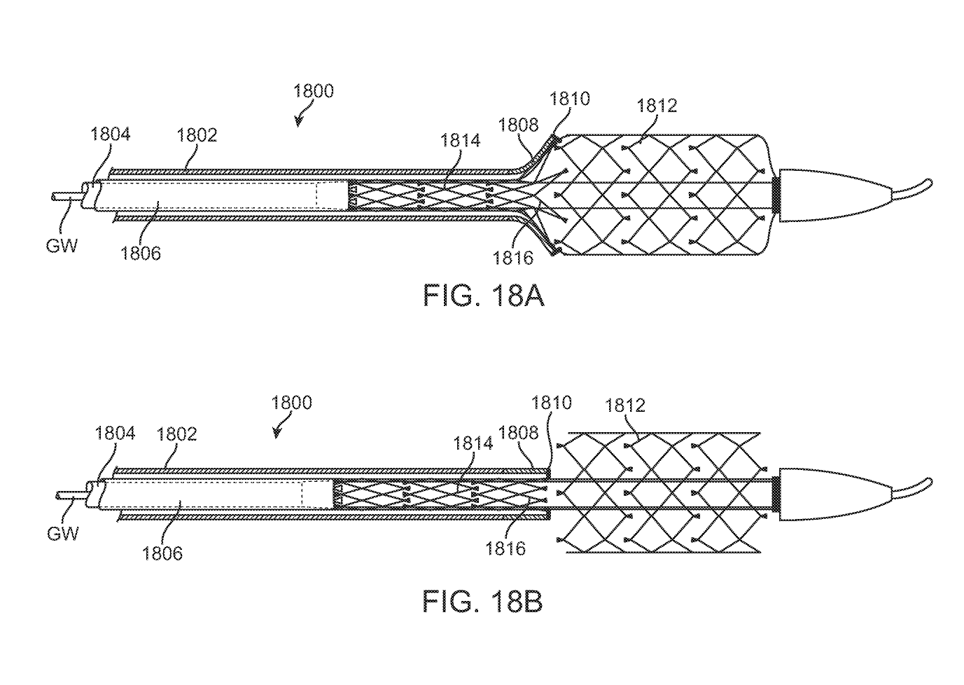

Another feature which some stent delivery catheters may include is a flange 1810 attached to the resilient section 1808 of outer sheath 1802, as seen in FIG. 18A. After the stents 1812 are expanded and the balloon is deflated, the resilient section 1808 collapses over the stents remaining with the catheter. Flange 1810 may then be used to help grab onto the remaining stents and pull them back proximally away from the deployed stents as the outer sheath is retracted proximally.

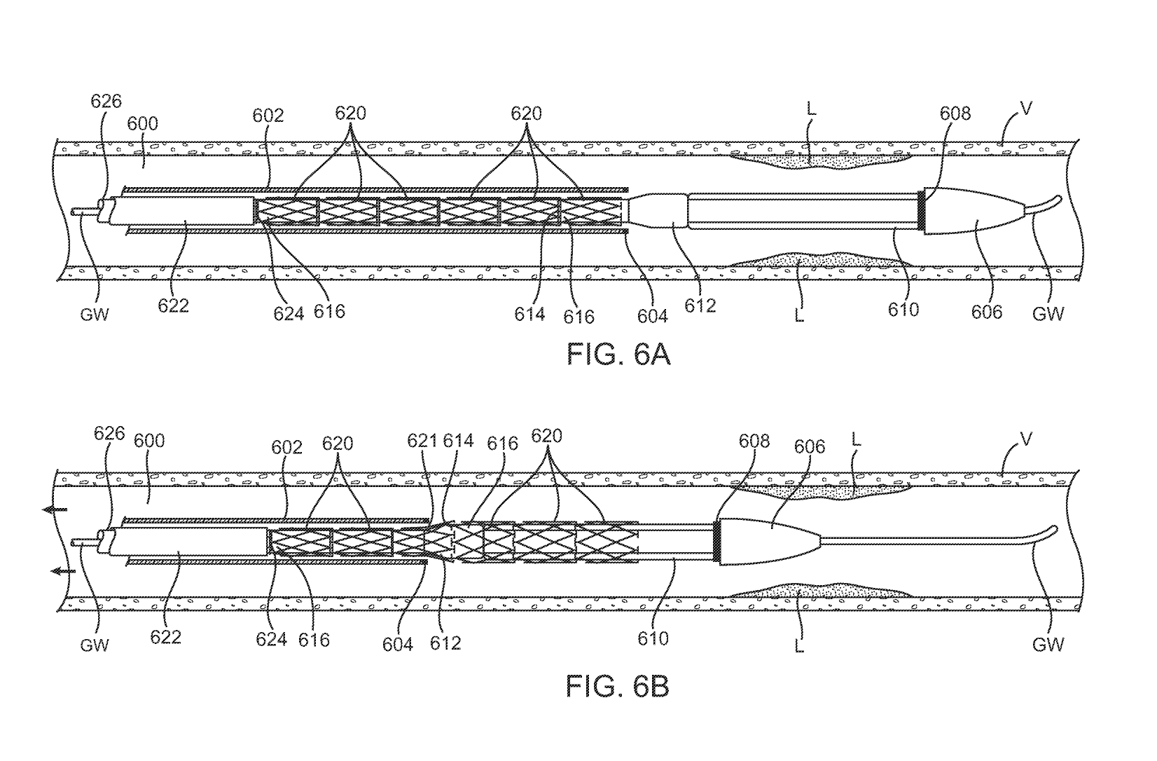

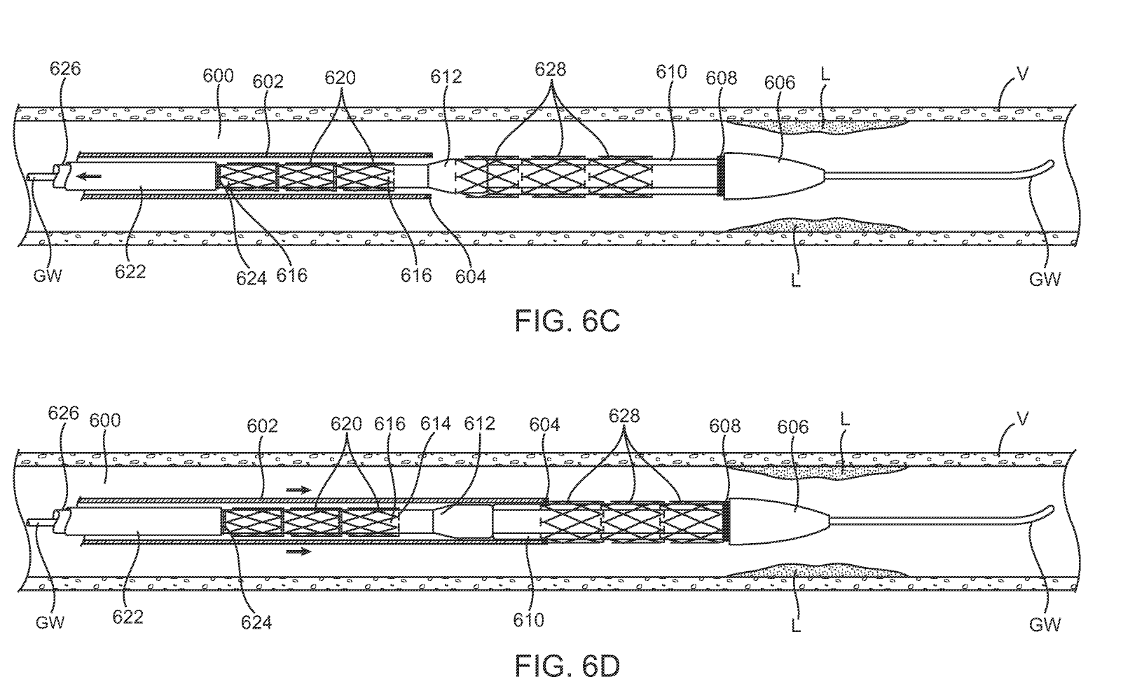

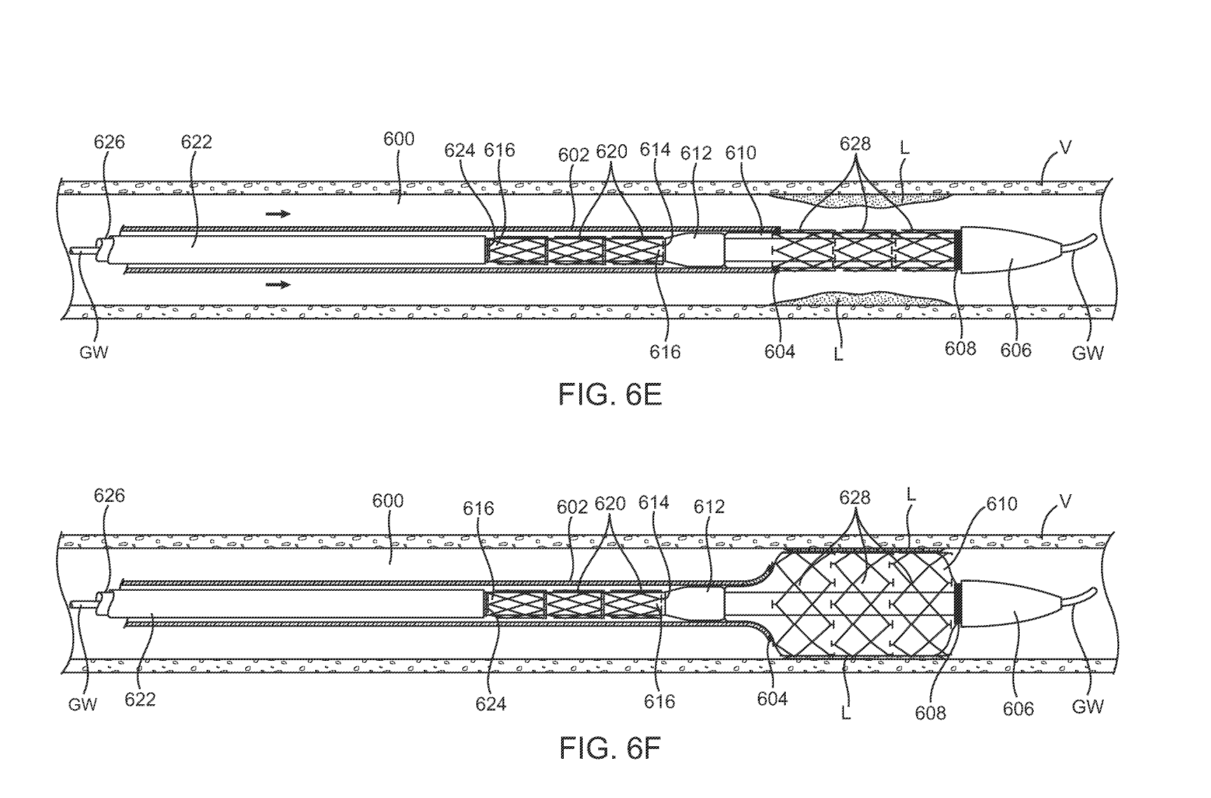

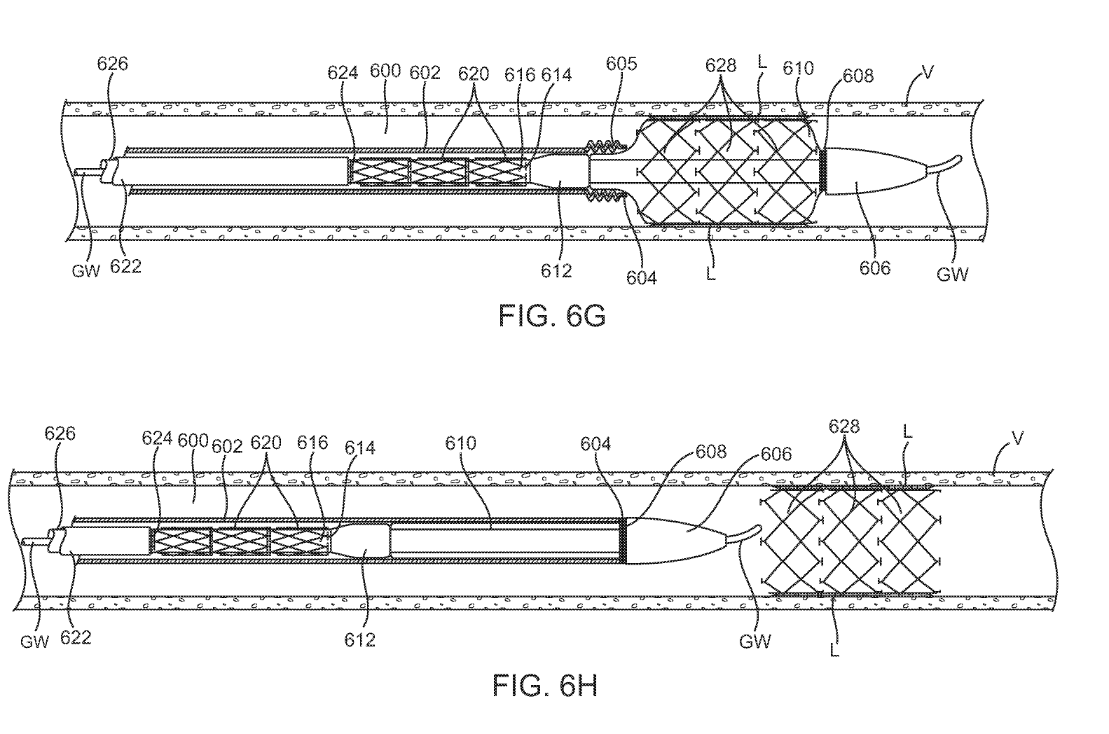

In another exemplary embodiment, a stent delivery catheter similar to catheter 20 of FIG. 1 also includes a transition ramp between the catheter inner shaft and the balloon. This ramp partially expands stent segments and allows stent segments to be selected for delivery and separated from the stent segments remaining with the delivery catheter. FIGS. 6A-6H illustrate this embodiment and how it may be used in a coronary stent procedure. Any of the features previously discussed above as well as the stent geometries previously described may be used with this delivery catheter.

In FIG. 6A, stent delivery catheter 600 is introduced into a coronary artery using standard catheterization techniques as previously described. Stent delivery catheter 600 is advanced over a guidewire GW so that a distal radiopaque marker/stent stop 608 adjacent to nose cone 606 is distal of the lesion L in the vessel V. The stent delivery catheter 600 includes a balloon 610 near the distal end of the catheter and a transition ramp 612 between a proximal section of inner shaft 626 and a distal section of inner shaft 626. The ramp 612 provides a smooth transition from the smaller outer diameter of the proximal section to the larger diameter of the distal section. Additional details are discussed below. A plurality of stent segments 620 are disposed over inner shaft 626 and they are releasably interlocked with one another. Each end of a stent segment 620 has a plurality of T-shaped ends 614 that are received in the space 616 between T-shaped ends on an adjacent stent segment 620 thereby releasably interlocking stent segments 620 together. Distal end of distal-most stent segment 620 is near the proximal end of ramp 612. An outer sheath 602 is disposed over at least some of the stents 620 and a radiopaque marker 604 is near the distal tip of outer sheath 602. Distal tip of outer sheath 620 is near proximal end of ramp 612. A stent moving tube 622 is disposed axially over the inner shaft 626 and also has a distal end with T-shaped ends 624 that releasably interlock with space 616 on the proximal-most stent segment 620, thereby permitting stent segments 620 to be moved proximally or distally as the moving tube 622 is retracted or advanced, respectively.

In FIG. 6B, inner shaft 626 and outer sheath 602 are retracted proximally while moving tube 622 is held stationary. Inner shaft 626 is retracted until radiopaque marker 608 is proximal of lesion L. This causes a selected number of stent segments to be advanced over ramp 612 and onto balloon 610. As stent segments 620 advance over ramp 612, they partially expand, thus space 616 increases, allowing T-shaped ends 614 to be released from space 616. Ramp 612 is sized so that its proximal end has an outer diameter that is approximately the same as the outer diameter of the inner shaft 626 and the distal end has an outer diameter that is substantially the same as the outer diameter of the unexpanded balloon 610. The ramp angle can vary over a wide range of angles from 5 to 75 degrees, more preferably from 20 to 60 degrees and even more preferably from 30 to 50 degrees. The ramp may have any length although often it may have a length equivalent to ½ to two stent segments or more. In FIG. 6B, three stent segments 620 are disposed over balloon 610 while a fourth stent segment 621 is partially expanded and lies over ramp 612. The rest of the stent segments 620 remain unexpanded and disposed over inner shaft 626.

In FIG. 6C, stent moving tube 622 is retracted proximally, thereby simultaneously retracting unselected stents 620. The distal-most stent 621 is released from the proximal-most stent 628 selected for delivery, therefore three stent segments 628 have been selected for delivery while three stent segments 620 remain with the deliver catheter. As stent 621 is retracted proximally into sheath 602, it is re-crimped back down to its unexpanded diameter.

In FIG. 6D, outer sheath 602 is advanced distally until it engages the stent segments 628 selected for delivery. The inner diameter of sheath 602 is sized so that it may be advanced over ramp 612 and balloon 610 while still engaging stent segments 628. Outer sheath 602 is advanced until the selected stent segments 628 are pushed up against stent stop/radiopaque marker 608.

Delivery catheter 600 is then advanced distally so that radiopaque marker 608 is distal to lesion L as seen in FIG. 6E. In FIG. 6F, balloon 610 is expanded so that stent segments 628 are also expanded into lesion L. A small gap exists between the distal end of outer sheath 602 and the proximal-most stent 628 selected for delivery. This gap is necessary so that stent segments 628 expand over a uniformly flat section of balloon 610, and not over a tapered portion of the balloon 610. In alternative embodiments, as seen in FIG. 6G, a resilient bellows 605 may be incorporated into the distal end of outer sheath 602. The bellows axially contracts as the balloon 610 is inflated, thereby automatically forming the required gap between the distal end of outer sheath 602 and the proximal-most stent 628 selected for delivery. Instead of the bellows 605, some embodiments may include the optional resilient outer sheath tip previously described with respect to FIGS. 5A-5H. In FIG. 6H, balloon 610 is deflated leaving stents 628 implanted at the site of lesion L. Delivery catheter 600 is withdrawn from expanded stents 628 and outer sheath is advanced so that its distal end is engaged with radiopaque marker 608. Delivery catheter 600 may then be repositioned within the vasculature and another stent or stents may be deployed at another lesion.

It should be understood that when the movement of the pusher tube, sheath, or stent segments is described in relation to other components of the delivery catheter of the invention, such movement is relative and will encompass moving some combination of the sheath, pusher tube, inner shaft or stent segments while keeping some of the other component(s) stationary.

Although the above is complete description of the preferred embodiments of the invention, various alternatives, additions, modifications and improvements may be made without departing from the scope thereof, which is defined by the claims.

What is claimed is:

1. A catheter for delivering a prosthesis to a target treatment site, the catheter comprising:
an inner shaft having a proximal end and a distal end;
an expansion member coupled to the inner shaft near the distal end;
a plurality of radially expandable prosthetic segments positionable over the expansion member, the plurality of radially expandable prosthetic segments releasably interlocked with one another while unexpanded and wherein adjacent pairs of the prosthetic segments are adapted to decouple from one another upon radial expansion of a distal prosthetic segment of the adjacent pair while a proximal prosthetic segment of the adjacent pair remains at least partially unexpanded;
an outer sheath axially movable relative to the expansion member and positionable at least partially over the plurality of radially expandable prosthetic segments to constrain expansion of a selectable number thereof; and
a segment mover axially movable relative to the expandable member and coupled to at least one of the plurality of radially expandable prosthetic segments, the segment mover adapted to retract one or more of the plurality of radially expandable prosthetic segments proximally relative to the expansion member when the one or more prosthetic segments is unexpanded.

2. The catheter of claim 1, further comprising a control mechanism coupled to the proximal end of the inner shaft.

3. The catheter of claim 2, wherein the control mechanism comprises an actuator adapted to move the outer sheath.

4. The catheter of claim 2, wherein the control mechanism comprises an actuator adapted to move the segment mover.

5. The catheter of claim 1, wherein the expansion member is expandable.

6. The catheter of claim 5, wherein the expansion member is a balloon.

7. The catheter of claim 1, wherein the plurality of prosthetic segments carry a therapeutic agent adapted to being released therefrom.

8. The catheter of claim 7, wherein the therapeutic agent comprises an anti-restenosis agent.

9. The catheter of claim 1, wherein each of the prosthetic segments has at least one locking element on a distal end thereof and at least one receptacle region on a proximal end thereof, the receptacle region being configured to capture the locking element of an adjacent prosthetic segment when the prosthetic segments are unexpanded so as to constrain axial movement of one prosthetic segment away from the other prosthetic segment, and wherein upon radial expansion of a prosthetic segment the receptacle region thereof releases the locking element of an adjacent unexpanded prosthetic segment.

10. The catheter of claim 9, wherein upon radial expansion of the prosthetic segment the receptacle region thereof is configured to change shape thereby releasing the locking element of the adjacent prosthetic segment.

11. The catheter of claim 9, wherein upon radial expansion of the prosthetic segment the receptacle region is configured to change positions relative to the adjacent prosthetic segment, thereby releasing the locking element of the adjacent prosthetic segment.

12. The catheter of claim 9, wherein the receptacle region is disposed between two locking elements on the same prosthetic segment.

13. The catheter of claim 9, wherein each locking element defines a receptacle region to capture a locking element on an adjacent prosthetic segment.

14. The catheter of claim 1, wherein at least one of the plurality of prosthetic segments comprises an axially extending member having an enlarged head region adapted to releasably interlock with a receptacle on an adjacent prosthetic segment.

15. The catheter of claim 14, wherein the enlarged head region is triangular shaped.

16. The catheter of claim 14, wherein the receptacle is formed by a space between two axially extending members each having an enlarged head region on an adjacent prosthetic segment.

17. The catheter of claim 14, wherein the receptacle widens upon expansion of the adjacent prosthetic segment.

18. The catheter of claim 1, wherein at least one of the plurality of prosthetic segments comprises one or more arms axially extending therefrom and adapted to releasably interlock with one or more arms axially extending from an adjacent prosthetic segment.

19. The catheter of claim 18, wherein the one or more axially extending arms release from one or more arms axially extending from an adjacent prosthetic segment upon expansion of the adjacent prosthetic segment.

20. The catheter of claim 18, wherein at least some of the arms are T-shaped.

21. The catheter of claim 18, wherein at least some of the arms are L-shaped.

22. The catheter of claim 1, wherein the inner shaft has a lumen disposed between the proximal and distal ends and adapted to accommodate a guidewire.

23. The catheter of claim 1, wherein the outer sheath comprises a resilient section near a distal end thereof, the resilient section adapted to expand as a prosthetic segment positioned therein is expanded by the expansion member and wherein the resilient section is adapted to collapse substantially back to its unexpanded configuration as the expansion member is collapsed.

24. The catheter of claim 23, wherein the resilient section is adapted to crimp any portion of a prosthetic segment disposed thereunder back to a substantially unexpanded configuration when the resilient section collapses.

25. The catheter of claim 23, wherein the resilient section comprises a plurality of fingers axially extending away from the outer sheath.

26. The catheter of claim 25, wherein the fingers are hinged.

27. The catheter of claim 25, wherein the fingers have a plurality of apertures extending therethrough.

28. The catheter of claim 1, wherein the sheath is adapted to collapse a partially expanded prosthetic segment back to a substantially unexpanded configuration by retraction of the partially expanded prosthetic segment into the sheath.

29. The catheter of claim 1, wherein the outer sheath comprises a flange near a distal end thereof, the flange adapted to engage a prosthetic segment as the outer sheath is retracted proximally so as to retract the prosthetic segment therewith.

30. The catheter of claim 1, wherein a portion of the outer sheath is reinforced so as to restrain expansion of at least a portion of the expansion member.

31. The catheter of claim 1, further comprising a crimping member positionable over a prosthetic segment and adapted to crimp the prosthetic segment to a reduced diameter when the crimping member is disposed thereover.

32. The catheter of claim 31, wherein the crimping member is disposed over the outer sheath.

33. The catheter of claim 31, wherein the crimping member comprises an o-ring.

34. The catheter of claim 31, wherein the crimping member comprises a tube slidably movable over the outer sheath.

35. The catheter of claim 5, further comprising an automatic separation mechanism coupled to the prosthetic segment moving tube such that the moving tube is adapted to retract as the expansion member expands thereby separating the unselected prosthetic segments from the selected number of prosthetic segments.

36. The catheter of claim 35, wherein the automatic separation mechanism comprises a piston mechanism coupled to the stent moving tube.

37. The catheter of claim 35, wherein the automatic separation mechanism comprises an actuator coupled to the stent moving tube.

38. The catheter of claim 35, wherein the expansion member and the automatic separation mechanism are fluidly coupled together.

39. The catheter of claim 5, further comprising an automatic separation mechanism coupled to the prosthetic segment moving tube such that the moving tube is adapted to retract as the as the expansion member contracts thereby separating the unselected prosthetic segments from the selected number of prosthetic segments.

40. The catheter of claim 39, wherein the automatic separation mechanism comprises a piston mechanism coupled to the prosthetic segment moving tube.

41. The catheter of claim 39, wherein the automatic separation mechanism comprises an actuator coupled to the prosthetic segment moving tube.

42. The catheter of claim 39, wherein the expansion member and the automatic separation mechanism are fluidly coupled together.

43. The catheter of claim 1, wherein the segment mover is adapted to advance the plurality of prosthetic segments distally as the moving tube is advanced distally.

44. The catheter of claim 1, wherein the segment mover is adapted to retract the interlocked plurality of prosthetic segments proximally as the mover is retracted proximally.

45. The catheter of claim 1, wherein the segment mover comprises a plurality of fingers axially extending therefrom and adapted to relesably interlock with fingers axially extending from an adjacent prosthetic segment.

46. The catheter of claim 1, wherein the segment mover comprises an axially extending member having an enlarged head region adapted to releasably interlock with a receptacle on an adjacent prosthetic segment.

47. The catheter of claim 1, further comprising:
a proximal section of the inner shaft having a first diameter;
a distal section of the inner shaft having a second diameter larger than the first diameter;
a ramp between the proximal section and the distal section and adapted to provide a transition from the first diameter to the second diameter,
wherein axially moving a prosthetic segment over the ramp partially expands the prosthetic segment from a first unexpanded diameter to a second partially expanded diameter, the prosthetic segment being adapted to decouple from adjacent prosthetic segments in the partially expanded diameter.

48. A catheter for delivering a prosthesis to a target treatment site, the catheter comprising:
an inner shaft having a proximal end, a distal end, a proximal section with a first diameter and a distal section with a second diameter larger than the first;
a ramp between the proximal and distal sections adapted to provide a transition from the first diameter to the second diameter;
an expandable member coupled to the inner shaft adjacent to the distal end;
a plurality of radially expandable prosthetic segments positionable over the expandable member,
the plurality of radially expandable prosthetic segments releasably interlocked with one another while unexpanded and wherein adjacent pairs of the prosthetic segments are adapted to decouple from one another upon radial expansion of a distal prosthetic segment of the adjacent pair while a proximal prosthetic segment of the adjacent pair remains at least partially unexpanded;
an outer sheath axially movable relative to the expansion member and positionable at least partially over the plurality of radially expandable prosthetic segments; and
a segment mover axially movable relative to the expandable member and coupled to at least one of the plurality of radially expandable prosthetic segments, the segment mover adapted to retract one or more of the plurality of radially expandable prosthetic segments proximally relative to the expandable member when the one or more prosthetic segments is unexpanded,
wherein axially moving a prosthetic segment over the ramp partially expands the prosthetic segment from a first unexpanded diameter to a second partially expanded diameter, the prosthetic segment being adapted to decouple from an adjacent prosthetic segment in the partially expanded diameter.

49. The catheter of claim 48, further comprising a control mechanism coupled to the proximal end of the inner shaft.

50. The catheter of claim 49, wherein the control mechanism comprises an actuator adapted to move the outer sheath.

51. The catheter of claim 49, wherein the control mechanism comprises an actuator adapted to move the segment mover.

52. The catheter of claim 48, wherein the expandable member is a balloon.

53. The catheter of claim 48, wherein the plurality of prosthetic segments carry a therapeutic agent adapted to being released therefrom.

54. The catheter of claim 53, wherein the therapeutic agent comprises an anti-restenosis agent.

55. The catheter of claim 48, wherein each of the prosthetic segments has at least one locking element on a distal end thereof and at least one receptacle region on a proximal end thereof, the receptacle region being configured to capture the locking element of an adjacent prosthetic segment when the prosthetic segments are unexpanded so as to constrain axial movement of one prosthetic segment away from the other prosthetic segment, and wherein upon radial expansion of a prosthetic segment the receptacle region thereof releases the locking element of an adjacent unexpanded prosthetic segment.

56. The catheter of claim 55, wherein upon radial expansion of the prosthetic segment the receptacle region thereof is configured to change shape thereby releasing the locking element of the adjacent prosthetic segment.

57. The catheter of claim 55, wherein upon radial expansion of the prosthetic segment the receptacle region is configured to change positions relative to the adjacent prosthetic segment, thereby releasing the locking element of the adjacent prosthetic segment.

58. The catheter of claim 55, wherein the receptacle region is disposed between two locking elements on the same prosthetic segment.

59. The catheter of claim 55, wherein each locking element defines a receptacle region to capture a locking element on an adjacent prosthetic segment.

60. The catheter of claim 48, wherein at least one of the plurality of prosthetic segments comprises an axially extending member having an enlarged head region adapted to releasably interlock with a receptacle on an adjacent prosthetic segment.

61. The catheter of claim 60, wherein the enlarged head region is triangular shaped.

62. The catheter of claim 60, wherein the receptacle is formed by a space between two axially extending members each having an enlarged head region on an adjacent prosthetic segment.

63. The catheter of claim 60, wherein the receptacle widens upon expansion of the adjacent prosthetic segment.

64. The catheter of claim 48, wherein at least one of the plurality of prosthetic segments comprises one or more arms axially extending therefrom and adapted to releasably interlock with one or more arms axially extending from an adjacent prosthetic segment.

65. The catheter of claim 64, wherein the one or more axially extending arms release from one or more arms axially extending from an adjacent prosthetic segment upon expansion of the adjacent prosthetic segment.

66. The catheter of claim 65, wherein at least some of the arms are T-shaped.

67. The catheter of claim 65, wherein at least some of the arms are L-shaped.

68. The catheter of claim 48, wherein the inner shaft has a lumen disposed between the proximal and distal ends and adapted to accommodate a guidewire.

69. The catheter of claim 48, wherein the segment mover is adapted to advance the plurality of prosthetic segments distally as the mover is advanced distally.

70. The catheter of claim 48, wherein the prosthetic segment mover is adapted to retract the plurality of prosthetic segments proximally as the mover is retracted proximally.

71. The catheter of claim 48, wherein the ramp has a proximal diameter that is substantially similar to the first diameter of the proximal section of the inner shaft.

72. The catheter of claim 48, wherein the ramp has a distal diameter that is substantially similar to the second diameter of the distal section of the inner shaft.

73. The catheter of claim 48, wherein a portion of the outer sheath is reinforced so as to restrain expansion of at least a portion of the expansion member

74. The catheter of claim 48, wherein the outer sheath comprises a resilient section near a distal end thereof, the resilient section adapted to expand as a prosthetic segment postioned therein is expanded by the expandable member and wherein the resilient section is adapted to collapse substantially back to its unexpanded configuration as the expandable member is collapsed.

75. The catheter of claim 74, wherein the resilient section is adapted to crimp any portion of a prosthetic segment disposed thereunder back to a substantially unexpanded configuration when the resilient section collapses.

76. The catheter of claim 74, wherein at least a portion of the resilient section is radiopaque.

77. The catheter of claim 74, wherein the resilient section comprises a plurality of fingers axially extending away from the outer sheath.

78. The catheter of claim 77, wherein the fingers are hinged.

79. The catheter of claim 77, wherein the fingers have a plurality of apertures extending therethrough.

80. The catheter of claim 48, wherein the sheath is adapted to collapse a partially expanded prosthetic segment back to a substantially unexpanded configuration by retraction of the partially expanded prosthetic segment into the sheath.

81. The catheter of claim 48, wherein the outer sheath comprises a flange near a distal end thereof, the flange adapted to engage a prosthetic segment as the outer sheath is retracted proximally so as to retract the prosthetic segment therewith.

82. The catheter of claim 48, wherein the outer sheath comprises a resilient section near a distal end thereof, the resilient section adapted to axially contract as the expandable member expands and wherein the resilient section expands substantially back to its uncontracted configuration as the expandable member is contracted.

83. The catheter of claim 82, wherein the resilient section comprises a bellows.

84. The catheter of claim 82, wherein the resilient section comprises a spring.

85. The catheter of claim 82, wherein the reslient section is adapted to allow a balloon taper to form when the resilient section axially contracts.

86. The catheter of claim 48, wherein the segment mover is releasably interlocked with at least one of the plurality of prosthetic segments.

87. The catheter of claim 48, wherein the segment mover comprises a plurality of fingers axially extending therefrom and adapted to releasably interlock with fingers axially extending from an adjacent prosthetic segment.

88. The catheter of claim 48, wherein the segment mover comprises an axially extending member having an enlarged head region adapted to releasably interlock with a receptacle on an adjacent prosthetic segment.

89. A method for deploying a prosthesis into a treatment site in a body lumen, the method comprising:
advancing a delivery catheter to the treatment site, the delivery catheter having a plurality of radially expandable prosthetic segments disposed thereon and at least partially covered by a sheath,
wherein the plurality of radially expandable prosthetic segments are releasably interlocked with one another while unexpanded;
selecting a number of prosthetic segments to deploy into the body lumen, the selected number of prosthetic segments having a length substantially traversing a length of a lesion at the treatment site, the selected number being less than the total number of prosthetic segments on the catheter;
radially expanding the selected number of prosthetic segments, wherein the selected number of prosthetic segments decouple from at least one other of the prosthetic segments which remains at least partially unexpanded on the delivery catheter during expansion of the selected number of prosthetic segments;
retracting within the catheter the at least one other unexpanded prosthetic segments away from the selected number of expanded prosthetic segments; and
removing the delivery catheter from the treatment site with the at least one other unexpanded prosthetic segments thereon, the selected number of expanded prosthetic segments being implanted at the treatment site.

90. The method of claim 89, wherein the prosthetic segments are expanded by expanding an expansion member on the delivery catheter, and wherein the at least one other unexpanded prosthetic segments are retracted after the expansion member is expanded.

91. The method of claim 89, wherein a middle prosthetic segment is disposed between the selected number of prosthetic segments to be expanded and the at least one other prosthetic segments constrained from expansion, the middle prosthetic segment having a proximal portion which is unexpanded and a distal portion which is partially expanded when the selected number of prosthetic segments are expanded.

92. The method of claim 91, wherein a distal end of the middle prosthetic segment is released from the selected number of prosthetic segments upon the expansion thereof, a proximal end of the middle prosthetic segment remaining connected to the at least one other prosthetic segments which remain unexpanded.

93. The method of claim 91, wherein the step of retracting comprises retracting the middle prosthetic segment into the sheath, the sheath crimping the distal portion of the middle prosthetic segment into an unexpanded shape.

94. The method of claim 89, wherein selecting comprises actuating a control mechanism adjacent to a proximal end of the delivery catheter.

95. The method of claim 89, wherein selecting comprises moving the outer sheath so as to expose the selected number of prosthetic segments from the outer sheath.

96. The method of claim 95, wherein moving the outer sheath comprises proximally retracting the outer sheath.

97. The method of claim 89, wherein radially expanding the selected number of prosthetic segments comprises inflating a balloon.

98. The method of claim 89, wherein radially expanding the selected number of prosthetic segments comprises moving an expansion member under the selected number of prosthetic segments causing radial expansion thereof.

99. The method of claim 98, wherein the expansion member comprises a substantially spherical head.

100. The method of claim 89, wherein radially expanding the selected number of prosthetic segments comprises flaring a distal end of the outer sheath as the prosthetic segments expand.

101. The method of claim 89, further comprising the step of radially contracting an expandable member.

102. The method of claim 101, wherein radially contracting comprises deflating a balloon.

103. The method of claim 101, wherein radially contracting the expandable member comprises collapsing a flared distal end of the outer sheath.

104. The method of claim 103, wherein collapsing the flared distal end of the outer sheath crimps at least a portion of a prosthetic segment disposed thereunder to a reduced diameter onto the delivery catheter.

105. The method of claim 89, wherein retracting within the catheter comprises moving the remaining prosthetic segments into the sheath.

106. The method of claim 89, wherein retracting comprises retracting a segment mover releasably coupled to the remaining prosthetic segments.

107. The method of claim 89, wherein retracting comprises retracting the outer sheath, the outer sheath having a flared distal section engaged with at least one of the unselected prosthetic segments.

108. The method of claim 105, wherein moving the remaining prosthetic segments comprises crimping a portion thereof back to a substantially unexpanded configuration.

109. The method of claim 89, further comprising advancing the remaining prosthetic segments toward a distal end of the delivery catheter.

110. The method of claim 109, wherein advancing the remaining prosthetic segments comprises pushing the remaining prosthetic segments distally with a segment mover connected thereto.

111. The method of claim 89, further comprising delivering a therapeutic agent to the treatment site, the therapeutic agent coupled to the prosthetic segments and adapted to being released therefrom.

112. The method of claim 111, wherein the therapeutic agent inhibits restenosis.

113. A method for deploying a prosthesis into a treatment site in a body lumen, the method comprising:
advancing a delivery catheter to the treatment site, the delivery catheter having a plurality of radially expandable prosthetic segments disposed thereon and at least partially covered by a sheath,
wherein the plurality of radially expandable prosthetic segments releasably interlock with one another while unexpanded;
selecting a number of prosthetic segments to deploy into the body lumen, the selected number of prosthetic segments having a length substantially traversing a length of a lesion at the treatment site, the selected number being less than the total number of prosthetic segments on the catheter;
partially expanding the selected number of prosthetic segments, wherein the selected number of prosthetic segments decouple from at least one other of the prosthetic segments which remains at least partially unexpanded on the delivery catheter during expansion of the selected number of prosthetic segments;
retracting within the catheter the at least one other partially unexpanded prosthetic segments away from the selected number of expanded prosthetic segments; and
radially expanding the selected number of prosthetic segments into the treatment site.

114. The method of claim 113, wherein the prosthetic segments are expanded by expanding an expansion member on the delivery catheter, and wherein the at least one other partially unexpanded prosthetic segments are retracted after the expansion member is expanded.

115. The method of claim 113, wherein a middle prosthetic segment is disposed between the selected number of prosthetic segments to be expanded and the at least one other prosthetic segments, the middle prosthetic segment having a proximal portion which is unexpanded and a distal portion which is partially expanded when the selected number of prosthetic segments are expanded.

116. The method of claim 115, wherein a distal end of the middle prosthetic segment is released from the selected number of prosthetic segments upon the expansion thereof, a proximal end of the middle prosthetic segment remaining connected to the at least one other prosthetic segments which remain partially unexpanded.

117. The method of claim 115, wherein the step of retracting comprises retracting the middle prosthetic segment into the sheath, the sheath crimping the distal portion of the middle prosthetic segment into an unexpanded shape.

118. The method of claim 113, wherein retracting comprises retracting any unselected prosthetic segments into the sheath.

119. The method of claim 118, wherein retracting the unselected prosthetic segments into the sheath crimps at least a portion of one of the segments into a substantially unexpanded configuration.

120. The method of claim 113, further comprising advancing the selected number of prosthetic segments toward a distal end of the delivery catheter prior to radial expansion thereof.

121. The method of claim 113, wherein selecting comprises advancing the selected number of prosthetic segments over an expandable member adjacent to a distal end of the delivery catheter.

122. The method of claim 113, wherein selecting comprises positioning an expandable member coupled to a distal end of the delivery catheter such that the selected number of prosthetic segments are positioned thereover.

123. The method of claim 122, wherein positioning comprises retracting the expandable member into the selected number of prosthetic segments.

124. The method of claim 113, wherein selecting comprises actuating a control mechanism adjacent to a proximal end of the delivery catheter.

125. The method of claim 113, wherein partially expanding comprises advancing the selected number of prosthetic segments over a ramped section of the delivery catheter.

126. The method of claim 113, wherein retracting any unselected prosthetic segments within the catheter comprises crimping at least one of the unselected prosthetic segments to a reduced profile.

127. The method of claim 113, further comprising advancing the outer sheath distally so as to engage and move at least one of the selected number of prosthetic segments distally.

128. The method of claim 113, wherein radially expanding the selected number of prosthetic segments comprises inflating a balloon disposed near a distal end of the delivery catheter.

129. The method of claim 113, wherein radially expanding the selected number of prosthetic segments comprises flaring a distal end of the outer sheath, the distal end of the sheath expanding with the selected prosthetic segments.

130. The method of claim 113, wherein radially expanding the selected number of prosthetic segments comprises axially compressing a distal portion of the outer sheath as an expandable member expands.

131. The method of claim 113, further comprising collapsing a flared distal portion of the outer sheath.

132. The method of claim 131, wherein collapsing the flared distal portion of the outer sheath crimps at least one prosthetic segment to a substantially unexpanded configuration reduced profile onto the delivery catheter.

133. The method of claim 113, further comprising retracting an expandable member into the outer sheath.

134. The method of claim 113, further comprising delivering a therapeutic agent to the lesion, the therapeutic agent coupled to the prosthetic segments and adapted to being released therefrom.

135. The method of claim 134, wherein the therapeutic agent inhibits restenosis.

136. The method of claim 113, further comprising contracting an expandable member so that it may be withdrawn from the selected prosthetic segments into the outer sheath.

137. The method of claim 113, further comprising contracting an expandable member so that it may be withdrawn from the selected prosthetic segments into the outer sheath.
